(19) United States
(12) Patent Application Publication
(10) Pub. No.: US 2019/0265270 A1
(43) Pub. Date: Aug. 29, 2019

(54) INFORMATION PROCESSING APPARATUS

(71) Applicant: Sony Corporation, Tokyo (JP)

(72) Inventors: Kosei YAMASHITA, Kanagawa (JP); Hidetoshi KABASAWA, Kanagawa (JP); Sho MURAKOSHI, Tokyo (JP); Tomohiro MATSUMOTO, Kanagawa (JP); Masahiro SEGAMI, Kanagawa (JP)

(21) Appl. No.: 16/349,171

(22) PCT Filed: Sep. 25, 2017
(86) PCT No.: PCT/JP2017/034515

§ 371 Date: May 10, 2019

(30) Foreign Application Priority Data
Nov. 11, 2016 (JP) 2016-220962

(51) Int. Cl. G01P 15/18; G01P 15/09; A61B 5/11

(52) U.S. Cl. CPC G01P 15/18; G01P 15/09; A61B 2562/0219; A61B 5/1123; A61B 5/1116

(57) ABSTRACT

An information processing system according to an embodiment of the present technology includes a controller unit. The controller unit calculates, on a basis of a dynamic acceleration component and a static acceleration component of a detection target moving within a space that are extracted from an acceleration in each direction of three axes of the detection target, a temporal change of the dynamic acceleration component with respect to the static acceleration component, and determines a motion of the detection target on a basis of the temporal change of the dynamic acceleration component.

### TECHNICAL FIELD

The present technology relates to an information processing apparatus that is applied to the technology of recognizing an activity of a user, for example.

### BACKGROUND ART

The activity recognition technology of recognizing an activity of a user by using a detected value of an acceleration sensor or the like mounted to a mobile apparatus or wearable apparatus carried or worn by the user has been developed (see, for example, Patent Literature 1). Such mobile apparatuses or wearable apparatuses include a mobile apparatus carried in a pocket of a pair of pants and a wrist-band-type wearable apparatus, for example, many of which are assumed to be carried while being substantially fixed to the body of a user.

### CITATION LIST

### Patent Literature

Patent Literature 1: Japanese Patent Application Laid-open No. 2016-6611

### DISCLOSURE OF INVENTION

### Technical Problem

In recent years, there has been a demand for the degree of freedom of the mountability of a sensor. A sensor in a mounting form in which the sensor is not fixed to the body of a user, e.g., a sensor of a neck-hanging type, detects a complicated motion including a pendulum motion of the sensor itself, in addition to the motion of the user. For that reason, it has been difficult to grasp a correct motion of the user.

In view of the circumstances as described above, it is an object of the present technology to provide an information processing apparatus capable of correctly grasping a motion of a detection target also in a case where a sensor in which a distance between the detection target and the sensor is variable is carried.

### Solution to Problem

An information processing apparatus according to an embodiment of the present technology includes a controller unit.

The controller unit calculates, on a basis of a dynamic acceleration component and a static acceleration component of a detection target moving within a space that are extracted from an acceleration in each direction of three axes of the detection target, a temporal change of the dynamic acceleration component with respect to the static acceleration component, and determines a motion of the detection target on a basis of the temporal change of the dynamic acceleration component.

In the information processing apparatus described above, since the controller unit is configured to calculate the temporal change of the dynamic acceleration component with respect to the static acceleration component of the acceleration and determine the motion of the detection target on the basis of the temporal change of the dynamic acceleration component, the motion of the detection target can be more correctly grasped.

The controller unit may include an arithmetic unit and a pattern recognition unit. The arithmetic unit calculates a normalized dynamic acceleration obtained by normalizing the dynamic acceleration component in a gravity direction. The pattern recognition unit determines the motion of the detection target on a basis of the normalized dynamic acceleration.

The arithmetic unit may further calculate a posture angle of the detection target on a basis of information related to an angular velocity about each of the three axes. In this case, the pattern recognition unit determines the motion of the detection target on a basis of the normalized dynamic acceleration and the posture angle.

The pattern recognition unit may be configured to determine an activity class of the detection target on a basis of the motion of the detection target.

The information processing apparatus may further include a detector unit that is attached to the detection target and detects the acceleration.

The detector unit may include an acceleration arithmetic unit. The acceleration arithmetic unit extracts the dynamic acceleration component and the static acceleration component for each direction of the three axes on a basis of a first detection signal having an alternating-current waveform corresponding to the acceleration and a second detection signal having an output waveform in which an alternating-current component corresponding to the acceleration is superimposed on a direct-current component.

The acceleration arithmetic unit may include an arithmetic circuit that extracts the static acceleration component from the acceleration on a basis of a difference signal between the first detection signal and the second detection signal.

The acceleration arithmetic unit may further include a gain adjustment circuit that adjusts gain of each signal such that the first detection signal and the second detection signal have an identical level.

The acceleration arithmetic unit may further include a correction circuit that calculates a correction coefficient on a basis of the difference signal and corrects one of the first detection signal and the second detection signal by using the correction coefficient.

The detector unit may be configured to be portable without being fixed to the detection target.

The detector unit may include a sensor element. The sensor element includes an element main body that includes a movable portion movable by reception of an acceleration, a piezoelectric first acceleration detector unit that outputs a first detection signal including information related to the acceleration in each direction of the three axes that acts on the movable portion, and a non-piezoelectric second acceleration detector unit that outputs a second detection signal including information related to the acceleration in each direction of the three axes that acts on the movable portion.

The second acceleration detector unit may include a piezoresistive acceleration detection element that is provided to the movable portion.

Alternatively, the second acceleration detector unit may include an electrostatic acceleration detection element that is provided to the movable portion.

### Advantageous Effects of Invention

As described above, according to the present technology, it is possible to correctly grasp a motion of a detector unit.

It should be noted that the effects described herein are not necessarily limited, and any of the effects described in the present disclosure may be produced.

### BRIEF DESCRIPTION OF DRAWINGS

FIG. 1 is a block diagram showing a schematic configuration of an activity pattern recognition system according to an embodiment of the present technology.

FIG. 2 is a schematic view for describing an application example of the activity pattern recognition system.

FIG. 3 is a configuration diagram of the activity pattern recognition system.

FIG. 4 is a block diagram of a basic configuration of a main part of the activity pattern recognition system.

FIG. 5 is a diagram for describing a time waveform obtained by the activity pattern recognition system.

FIG. 6 is a circuit diagram showing a configuration example of an acceleration arithmetic unit in a detector unit (inertial sensor) used in the activity pattern recognition system.

FIG. 7 is a schematic perspective view of the front surface side of an acceleration sensor element in the inertial sensor.

FIG. 8 is a schematic perspective view of the back surface side of the acceleration sensor element.

FIG. 9 is a plan view of the acceleration sensor element.

FIG. 10A is a schematic sectional side view for describing a state of a motion of a main part of the sensor element, which shows a state where accelerations are not applied.

FIG. 10B is a schematic sectional side view for describing a state of a motion of the main part of the sensor element, which shows a state where an acceleration along an x-axis direction occurs.

FIG. 100 is a schematic sectional side view for describing a state of a motion of the main part of the sensor element, which shows a state where an acceleration along a z-axis direction occurs.

FIG. 11 is a circuit diagram showing a configuration example of the acceleration arithmetic unit in the inertial sensor.

FIG. 12 is a diagram showing a processing block for a one-axis direction in the acceleration arithmetic unit.

FIG. 13 is a diagram for describing output characteristics of a plurality of acceleration sensors in different detection methods.

FIG. 14 is a diagram for describing an action of the acceleration arithmetic unit.

FIG. 15 is a diagram for describing an action of the acceleration arithmetic unit.

FIG. 16 is a diagram for describing an action of the acceleration arithmetic unit.

FIG. 17 is a diagram for describing an action of the acceleration arithmetic unit.

FIG. 18 is a diagram for describing an action of the acceleration arithmetic unit.

FIG. 19 is a diagram for describing an action of the acceleration arithmetic unit.

FIG. 20 is a flowchart showing an example of a processing procedure of the acceleration arithmetic unit.

FIG. 21 is a flowchart for describing an operation example of the activity pattern recognition system.

### MODE(S) FOR CARRYING OUT THE INVENTION

Hereinafter, an embodiment according to the present technology will be described with reference to the drawings. The present technology is applicable to a so-called activity recognition system or the like and measures a kinematic physical quantity of a detection target on the basis of information from a sensor carried by a human or another moving object as the detection target and records and displays the kinematic physical quantity, e.g., the history of an activity of the detection target.

[General Outline of Apparatus]

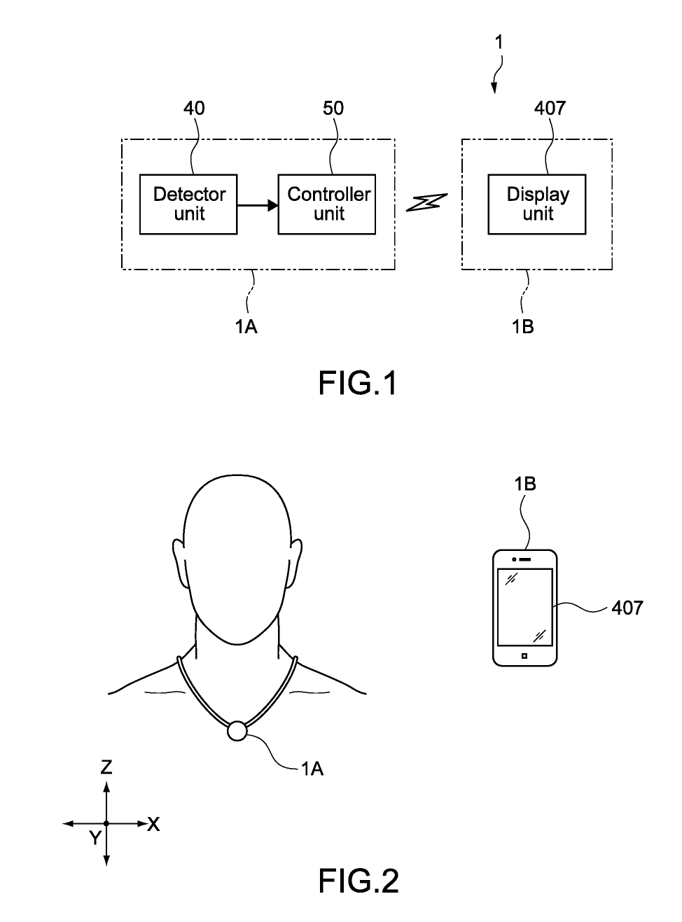

FIG. 1 is a block diagram showing a schematic configuration of an activity pattern recognition system according to an embodiment of the present technology. FIG. 2 is a schematic view for describing an application example of the activity pattern recognition system.

As shown in FIG. 1, an activity pattern recognition system 1 of this embodiment includes a sensor device 1A and a terminal device 1B, the sensor device 1A including a detector unit 40 and a controller unit 50, the terminal device 1B including a display unit 407. The activity pattern recognition system 1 is configured to record and display, for example, an activity history of a detection target as a kinematic physical quantity of the detection target that is moving within a space.

The sensor device 1A is configured to be portable without being fixed to a detection target. The terminal device 1B is configured to be communicable with the sensor device 1A wirelessly or wiredly and is typically constituted of a portable information terminal such as a smartphone, a mobile phone, or a laptop PC (personal computer).

In this embodiment, the sensor device 1A is used to detect the motion of the detection target, but the detector unit and the controller unit mounted to the sensor device 1A may be mounted to the terminal device 1B. For example, a single smartphone can record and display an activity history or the like of the detection target, which is obtained on the basis of the detection of a motion of the detection target and a detection result thereof.

In this embodiment, for example, as shown in FIG. 2, the pendant head of a pendant 3 worn around the neck of a user as a detection target is the sensor device 1A. The sensor device 1A is carried by the user without being fixed such that a distance from the detection target is variable while swinging along the motion of the user. The sensor device 1A is configured to extract a kinematic physical quantity of the detection target at each predetermined point of time or continuously and transmit the kinematic physical quantity to the terminal device 1B. For example, in this embodiment, information (activity history) in which an activity class as a kinematic physical quantity, position information, and point of time information are associated with one another is transmitted from the sensor device 1A to the terminal device 1B.

The terminal device 1B is configured to record and notify the user of the activity class, the position information, and the point of time information, which are acquired from the sensor device 1A. Examples of the activity class include a walking action, a running action, a rest state, a jumping action, getting in or out of a train, getting in or out of an elevator, an escalator, or the like, ascending, descending, going up and down on stairs, a state during doing sports, and a working state of the user. The information transmitted to the terminal device 1B is recorded in the terminal device 1B and is capable of being displayed such that the user can visually recognize the information in a desired display form.

The sensor device 1A includes a casing, and the detector unit 40 and the controller unit 50 housed in the casing.

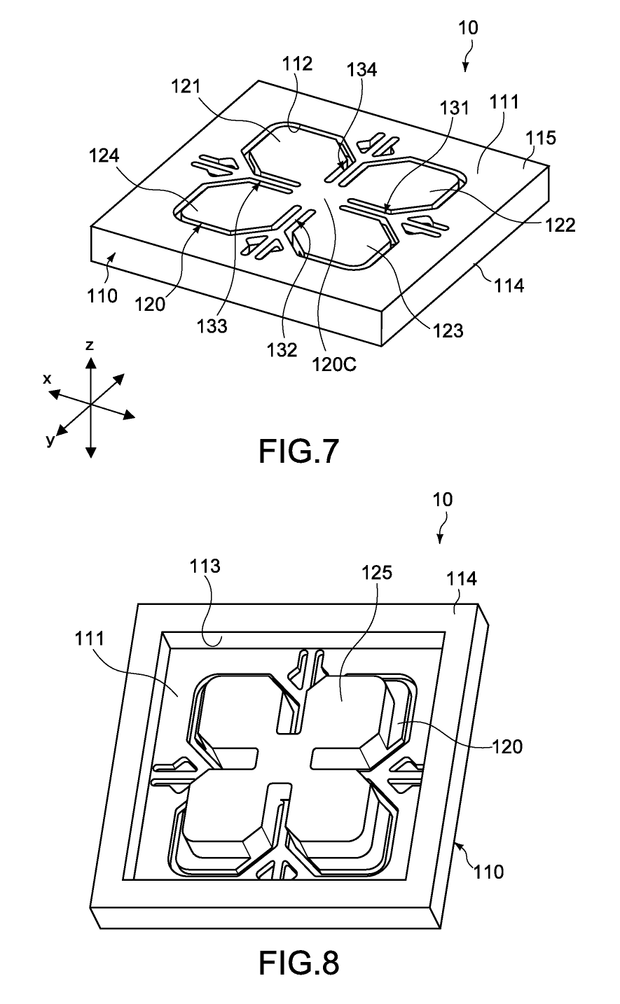

The detector unit 40 detects velocity-related information that is related to temporal changes in velocities in directions of three orthogonal axes (x axis, y axis, and z axis in FIG. 7) in a local coordinate system and angular velocities.

The controller unit 50 calculates the kinematic physical quantity of the user from the detected velocity-related information and angular velocities and generates and outputs the kinematic physical quantity as a control signal. Specifically, in this embodiment, the controller unit 50 detects an activity pattern of the user from the velocity-related information and the angular velocity information, determines this activity pattern by using a determination model generated in advance, to be classified (pattern recognition).

The method of carrying the sensor device 1A is not limited to this embodiment. For example, the sensor device may be mountable to a neck-hanging holder. Further, the sensor device 1A may be carried in a breast pocket of a shirt or a bag always carried by the user. Further, a function of the sensor device may be integrated in a portable terminal such as a smartphone.

The terminal device 1B includes the display unit 407 and is capable of displaying the activity history or the like of the user on the display unit 407 on the basis of the control signal.

Hereinafter, details of the activity pattern recognition system 1 according to this embodiment will be described.

[Basic Configuration]

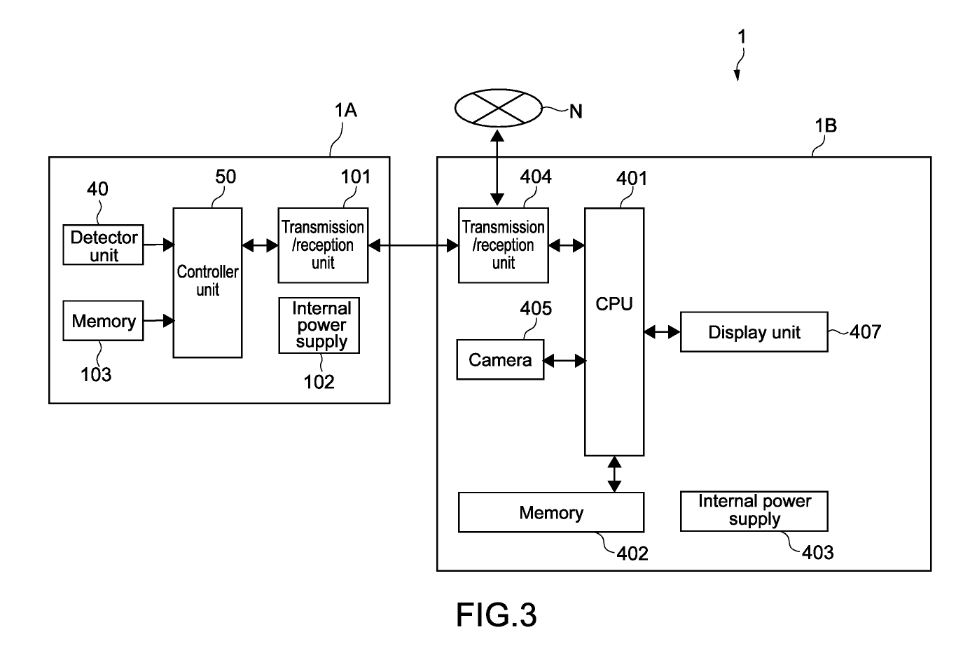

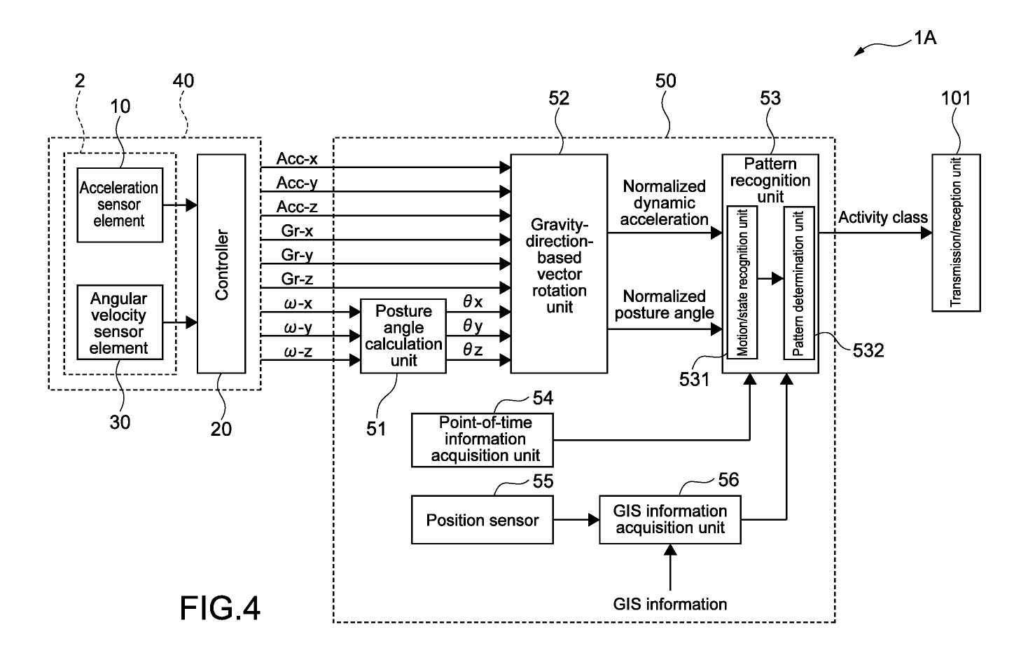

FIG. 3 is a system configuration diagram of the activity pattern recognition system 1, and FIG. 4 is a block diagram of a basic configuration of a main part thereof. The activity pattern recognition system 1 includes the sensor device 1A and the terminal device 1B.

(Sensor Device)

The sensor device 1A includes the detector unit 40, the controller unit 50, a transmission/reception unit 101, an internal power supply 102, a memory 103, and a power supply switch (not shown in the figure).

The detector unit 40 is an inertial sensor including an inertial sensor unit 2 and a controller 20.

The inertial sensor unit 2 includes an acceleration sensor element 10 and an angular velocity sensor element 30. The acceleration sensor element 10 detects the accelerations in the directions of the three orthogonal axes (x axis, y axis, and z axis in FIG. 7) in the local coordinate system. The angular velocity sensor element 30 detects the angular velocities about the three axes. The controller 20 processes an output from the inertial sensor unit 2.

In the inertial sensor unit 2 of this embodiment, the acceleration sensor and the angular velocity sensor for each axis are separately constituted, but the present technology is not limited thereto. The acceleration sensor and the angular velocity sensor may be a single sensor that is capable of simultaneously detecting accelerations and angular velocities in the three-axis directions. Further, a configuration in which the angular velocity sensor element 30 is not provided and an angular velocity is detected by using the acceleration sensor element 10 may also be provided.

In the detector unit 40, dynamic acceleration components (Acc-x, Acc-y, Acc-z), static acceleration components (Gr-x, Gr-y, Gr-z), and angular velocity signals (ω-x, ω-y, ω-z) in the local coordinate system, which are acquired in predetermined sampling periods, are calculated as the velocity-related information by the controller 20 on the basis of detection results of the inertial sensor unit 2, and are sequentially output to the controller unit 50.

In the detector unit 40, an acceleration detection signal including dynamic acceleration components and static acceleration components about the three axes of the sensor device 1A, which are detected from the acceleration sensor element 10, is separated into the dynamic acceleration components (Acc-x, Acc-y, Acc-z) and the static acceleration components (Gr-x, Gr-y, Gr-z) by the controller 20. The configuration of the acceleration sensor element 10 and the separation of the dynamic velocity components and the static acceleration components, which is performed by the controller 20, will be described later in detail.

Further, in the detector unit 40, angular velocity signals about the three axes (ω-x, ω-y, ω-z) are each calculated by the controller 20 on the basis of angular velocity detection signals about the three axes (Gyro-x, Gyro-y, Gyro-z) of the user U (sensor device 1A), which are detected from the angular velocity sensor element 30. The angular velocity sensor element 30 detects angular velocities about the x, y, and z axes (hereinafter, also referred to as angular velocity components in local coordinate system), respectively. For the angular velocity sensor element 30, a vibration type gyro sensor is typically used. In addition thereto, a rotary top gyro sensor, a laser ring gyro sensor, a gas rate gyro sensor, and the like may be used.

The controller unit 50 calculates, on the basis of the dynamic acceleration components and the static acceleration components of the detection target, which are extracted from the accelerations in the three-axis directions of the detection target (pendant 3) that moves within a space, a temporal change of the dynamic acceleration components with respect to the static acceleration components, and determines a motion of the detection target on the basis of the temporal change of the dynamic acceleration components.

In this embodiment, the controller unit 50 classifies activity patterns of the user and determines activity classes by using pattern recognition, in which a learning model obtained by supervised learning is used, on the basis of the velocity-related information including the dynamic acceleration components and static acceleration components output from the detector unit 40 and the angular velocity signals.

Examples of a learning method of the supervised learning include learning methods using a learning model such as template matching, NN (Neural Network), or HMM (Hidden Markov Model). In the supervised learning, information called the label of “correct” that indicates to which class the learning data of each pattern (data utilized in learning) belongs is given, and the learning data of the category to which the class belongs (is caused to belong) is learned for each class.

In the supervised learning, learning data to be used for learning is prepared for each category determined in advance, and a learning model to be used for learning (learning model by which learning data of each category is learned) is also prepared for each category determined in advance. In the pattern recognition using a learning model obtained by the supervised learning, for certain data to be recognized, the label of “correct” of a template that is most matched with the certain data to be recognized is output as a recognition result. In the pattern recognition processing using the learning model, teaching data as a set of input data and output data that is to be subjected to learning processing is prepared in advance.

The controller unit 50 includes a posture angle calculation unit 51, a vector rotation unit 52 (arithmetic unit), a pattern recognition unit 53, a point-of-time information acquisition unit 54, a position sensor 55, and a GIS information acquisition unit 56.

The posture angle calculation unit 51 calculates rotational angle components (θₓ, θ_{y}, θ_{z}) from the angular velocity components (ωₓ, ω_{y}, ω_{z}) in the local coordinate system, which are output from the angular velocity sensor element 30, and outputs the rotational angle components (θₓ, θ_{y}, θ_{z}) to the vector rotation unit 52.

The vector rotation unit 52 performs vector rotation and normalization on the input dynamic acceleration components (Acc-x, Acc-y, Acc-z) and rotational angle components (θₓ, θ_{y}, θ_{z}) with the gravity direction being set as a reference, calculates normalized dynamic accelerations, which are dynamic accelerations (motion accelerations) having no influence of the gravity, and normalized posture angles, which are posture angles having no influence of the gravity, and outputs them to the pattern recognition unit 53. The normalized dynamic accelerations and the normalized posture angles are information related to the motion of the user, in which a component related to the motion such as swing of the sensor device 1A itself is substantially cancelled.

In the calculation of the normalized dynamic accelerations, the vector rotation unit 52 may convert the dynamic acceleration components (Acc-x, Acc-y, Acc-z), which are output from the detector unit 40, into dynamic acceleration components (Acc-X, Acc-Y, Acc-Z) in a global coordinate system (X, Y, and Z axes in FIG. 2) direction in a real space. In this case, the rotational angle components (θₓ, θ_{y}, θ_{z}) input to the vector rotation unit 52 may be referred to. Further, in the calculation of the rotational angle components (θₓ, θ_{y}, θ_{z}), for example, calibration processing may be executed when the detector unit 40 remains motionless. With this configuration, the rotational angle of the detector unit 40 with respect to the gravity direction can be accurately detected.

The pattern recognition unit 53 detects a motion or an activity pattern of the user on the basis of the normalized dynamic accelerations and the normalized posture angles, and classifies the activity pattern of the user U to determine an activity class. Information in which the class (activity class) of the activity pattern that is the determined kinematic physical quantity, point of time information, and position information are associated with one another is transmitted to the transmission/reception unit 101.

The point-of-time information acquisition unit 54 acquires information of a point of time, information of a day of week, information of holiday, information of date, and the like, which are obtained when the detector unit 40 of the sensor device 1A performs detection, and outputs those pieces of information to the pattern recognition unit 53.

The position sensor 55 continuously or intermittently acquires position information indicating a place where the user is (hereinafter, current place). For example, position information of the current place is expressed by latitude, longitude, altitude, and the like. The position information of the current place acquired by the position sensor 55 is input to the GIS information acquisition unit 56.

The GIS information acquisition unit 56 acquires GIS (Geographic Information System) information. Additionally, the GIS information acquisition unit 56 detects the attribute of the current place by using the acquired GIS information. The GIS information includes, for example, map information and various additional information obtained by satellites, a field survey, and the like. The GIS information acquisition unit 56 expresses the attribute of the current place by using, for example, identification information called a geo category code. The geo category code is a classification code for classifying the types of information related to the place and is set depending on, for example, the type of a construction, the shape of a land, geographical characteristics, regionality, and the like.

The GIS information acquisition unit 56 refers to the acquired GIS information, identifies the current place, a construction around the current place, and the like, extracts a geo category code corresponding to that construction and the like, and outputs the geo category code to the pattern recognition unit 53.

The pattern recognition unit 53 includes a motion/state recognition unit 531 and an activity pattern determination unit 532. Here, the “motion/state” means an activity performed by the user for a relatively short time of about several seconds to several minutes. Examples of the motion include actions such as walking, running, jumping, resting, temporary stop, change in posture, ascending, and descending. Examples of the state include being in/on a train, an escalator, an elevator, a bicycle, a car, stairs, a sloping road, and a flat land. The “activity” is a living activity performed by the user with time longer than the time of the “motion/state”. Examples of the activity include having a meal, shopping, sports, working, and moving to a destination.

The motion/state recognition unit 531 detects an activity pattern by using the input normalized dynamic accelerations and normalized posture angles and inputs the activity pattern to the activity pattern determination unit 532.

The activity pattern is input from the motion/state recognition unit 531, the geo category code is input from the GIS information acquisition unit 56, and the point of time information is input from the point-of-time information acquisition unit 54, to the activity pattern determination unit 532. When those pieces of information are input, the activity pattern determination unit 532 determines the class of the activity pattern by using determination processing based on the learning model. The activity pattern determination unit 532 generates, as a control signal, information in which the class of the activity pattern (activity class), the position information, the point of time information, and the like are associated with one another, and outputs the control signal to the transmission/reception unit 101.

In the learning model determination, a determination model for determining the activity pattern is generated by using a machine learning algorithm, and an activity pattern corresponding to the input data is determined by using the generated determination model.

For the machine learning algorithm, for example, a k-means method, a Nearest Neibor method, SVM (Support Vector Maching), HMM (Hiden Markov Model), Boosting, Deep learning, and the like are utilizable.

The transmission/reception unit 101 includes a communication circuit and an antenna, for example, and constitutes an interface for communicating with the terminal device 1B (transmission/reception unit 404). The transmission/reception unit 101 is configured to be capable of transmitting an output signal including the control signal including the information in which the activity class, the position information, the point of time information, and the like are associated with one another and which is determined in the controller unit 50, to the terminal device 1B. Further, the transmission/reception unit 101 is configured to be capable of receiving setting information of the controller unit 50, which is transmitted from the terminal device 1B, and the like.

The communication performed between the transmission/reception unit 101 and the transmission/reception unit 404 of the terminal device 1B may be wireless or wired. The wireless communication may be communication using an electromagnetic wave (including infrared rays) or communication using an electric field. For a specific method, a communication method using a band ranging from several hundreds of MHz (megahertz) to several GHz (gigahertz), such as “Wi-Fi (registered trademark)”, “Zigbee (registered trademark)”, “Bluetooth (registered trademark)”, “Bluetooth Low Energy”, “ANT (registered trademark)”, “ANT+ (registered trademark)”, or “EnOcean (registered trademark)”, can be exemplified. Proximity wireless communication such as NFC (Near Field Communication) may also be used.

The internal power supply 102 supplies power necessary to drive the sensor device 1A. For the internal power supply 102, a power storage element such as a primary battery or a secondary battery may be used. Alternatively, an energy harvesting technique including a power-generating element for vibration power generation, solar power generation, or the like and parasitic means may be used. In particular, in this embodiment, since the detection target having a motion is a measurement target, an energy harvesting device such as a vibration power generation device is suitable for the internal power supply 102.

The memory 103 includes a ROM (Read Only Memory), a RAM (Random Access Memory), and the like and stores programs for executing control of the sensor device 1A by the controller unit 50, such as a program for generating the trajectory image signal (control signal) from the velocity-related information, various parameters, or data.

(Terminal Device)

The terminal device 1B is typically constituted of a portable information terminal and includes a CPU 401, a memory 402, an internal power supply 403, the transmission/reception unit 404, a camera 405, a position information acquisition unit (GPS (Global Positioning System) device) 406, and the display unit 407.

The CPU 401 controls the entire operation of the terminal device 1B. The memory 402 includes a ROM, a RAM, and the like and stores programs for executing control of the terminal device 1B by the CPU 401, various parameters, or data. The internal power supply 403 is for supplying power necessary to drive the terminal device 1B and is typically constituted of a chargeable/dischargeable secondary battery.

The transmission/reception unit 404 includes a communication circuit capable of communicating with the transmission/reception unit 101 and an antenna. The transmission/reception unit 404 is further configured to be capable of communicating with another portable information terminal, a server, and the like by using wireless LAN or a 3G or 4G network N for mobile communication.

The display unit 407 is constituted of, for example, an LCD (Liquid Crystal Display) or an OLED (Organic Light Emitting Diode) and displays GUIs (Graphic User Interface) of various menus, applications, or the like. Typically, the display unit 407 includes a touch sensor and is configured to be capable of inputting predetermined setting information to the sensor device 1A via the CPU 401 and the transmission/reception unit 404 by a touch operation of the user.

The activity history or the like of the user is displayed on the display unit 407 on the basis of the control signal from the sensor device 1A, which is received via the transmission/reception unit 404.

As in this embodiment, in a case where the sensor device 1A is of a neck-hanging type, a pendulum motion and other complicated motions are caused on the sensor device 1A itself along with the motion of the user, and the sensor device 1A detects, in addition to the motion of the human, complicated motions including the pendulum motion of the detector unit itself. In this embodiment, when the dynamic accelerations and the posture angles are normalized in the gravity direction, the normalized dynamic accelerations and normalized posture angles in which the motion of the sensor device 1A itself is substantially cancelled can be obtained.

In other words, assuming that the tilt of the sensor device 1A and the gravity direction have a high correlation, the gravity direction captured by the sensor device 1A becomes swing of the posture of the sensor device 1A as it is. Thus, when a gravitational acceleration component (static acceleration component) is subtracted from a detection acceleration detected by the sensor device 1A, and a result thereof is normalized in the gravity direction, the normalized dynamic acceleration in which the motion of the sensor device 1A itself is substantially cancelled can be obtained.

Therefore, the motion of the user can be correctly grasped from the normalized dynamic accelerations and normalized posture angles in which the motion of the sensor device 1A itself is substantially cancelled. Additionally, the activity pattern to be detected by using the normalized dynamic accelerations and the normalized posture angles becomes an activity pattern including a lot of motion components of the user, which facilitates the pattern recognition and enables highly accurate pattern recognition.

Further, while an acceleration component of the pendulum motion, which is the regular motion of the sensor device 1A, is left in the normalized dynamic acceleration described above, this acceleration component of the pendulum motion can be cancelled as noise in the pattern recognition. In other words, while the motion of the sensor device 1A itself includes an acceleration, that motion is impossible as the motion of a human or the user if only the posture of the sensor device 1A is considered. Thus, by the pattern recognition, the acceleration component of the pendulum motion of the sensor device 1A can be cancelled as noise.

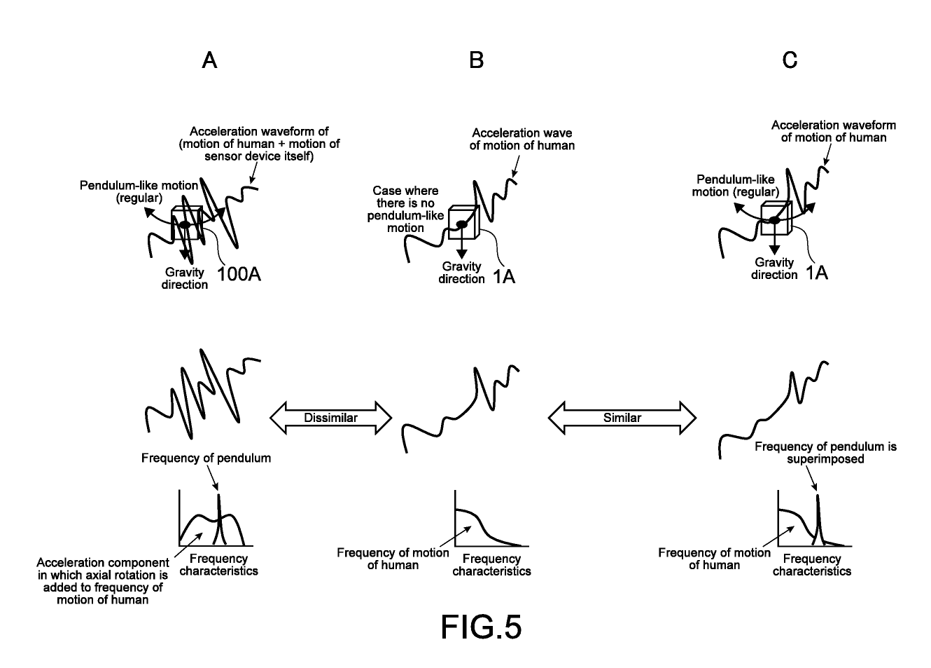

Next, a time waveform obtained by normalizing the dynamic acceleration components as in this embodiment will be described with reference to FIG. 5 in comparison with a comparison example.

[Time Waveform]

Each diagram of FIG. 5 shows, for example, a time waveform or the like of a detection acceleration in the X-axis direction, which is detected by an acceleration sensor element when the user carries a sensor device including the acceleration sensor element.

FIG. 5A is a comparison example and shows a case where the user carries a sensor device 100A hung around the neck without being fixed and the user is moving. Here, the dynamic acceleration component extracted from the detection acceleration detected by the sensor device as in this embodiment is not normalized. In this case, a detection acceleration in which complicated motions including a pendulum motion of the sensor device 100A itself and the motion of the user are combined is detected by the sensor device 100A, and FIG. 5A shows such a time waveform with an irregular wavy line. Further, a graph in the lower part of the figure shows frequency characteristics of the acceleration detected by the sensor device 100A. It is found from this graph that the acceleration detected by the sensor device 100A includes an acceleration component in which an axial rotation is added to the frequencies of the pendulum motion and the motion of the user.

FIG. 5B is a comparison example and shows a case where the user carries the sensor device 1A fixed to the body and the user is moving, in which swing of the pendulum motion or the like of the sensor device 1A itself does not occur. An irregular wavy line shown in FIG. 5B is a time waveform of the acceleration related to the motion of the user. Here, shown is a time waveform of the acceleration in a case where the dynamic acceleration component extracted from the detection acceleration detected from the acceleration sensor element 10 of the sensor device 1A is subjected to vector rotation and normalization. As compared to FIG. 5A, FIG. 5B is different in that the sensor device 1A is fixed and that the dynamic acceleration component is extracted from the acceleration detection signal and is subjected to vector rotation and normalization in the gravity direction. Further, a graph in the lower part of the figure shows frequency characteristics of the acceleration detected by the sensor device 1A and frequency characteristics related to the motion of the user.

FIG. 5C is an example according to this embodiment and shows a case where the user carries the sensor device 1A hung around the neck without being fixed and the user is moving, in which complicated motions including a pendulum motion of the sensor device 1A itself are generated in the sensor device 1A. An irregular wavy line shown in FIG. 5C is a time waveform of the normalized dynamic acceleration in which a dynamic acceleration component is subjected to vector rotation and normalization, the dynamic acceleration component being extracted from a detection acceleration, which is detected by the acceleration sensor element 10 and in which the complicated motions including the pendulum motion of the sensor device 1A itself and the motion of the user are combined. As compared to FIG. 5A, FIG. 5C is different in that the dynamic acceleration component is extracted from the acceleration detection signal and is subjected to vector rotation to be normalized in the gravity direction. Further, a graph in the lower part of the figure shows frequency characteristics of the normalized dynamic acceleration. It is found from this graph that the frequency of the normalized dynamic acceleration includes the frequencies related to the pendulum motion and the motion of the user.

As shown in FIGS. 5A and 5B, the respective time waveforms are dissimilar to each other. In contrast to this, as shown in FIGS. 5B and 5C, the respective time waveforms are similar to each other and are further, in the frequency characteristics, different from each other only in that the frequency of the pendulum motion is superimposed in FIG. 5C, and have substantially the same frequency characteristics related to the motion of the user.

With this configuration, the following is found: it is difficult to perform pattern recognition by the machine learning in the case of FIG. 5A in which the processing of normalizing the extracted dynamic acceleration component is not performed, whereas it is easy to perform pattern recognition by the machine learning in the case of FIG. 5C in which the processing of normalizing the extracted dynamic acceleration component is performed.

Therefore, the activity pattern detected on the basis of the normalized dynamic acceleration becomes an activity pattern including a lot of motion components of the user, which facilitates the pattern recognition and enables highly accurate pattern recognition.

In such a manner, in this embodiment, even if the sensor device 1A is not fixed to the body of the user and is carried by the user such that a distance between the sensor device 1A and the user is variable, the motion of the user can be substantially correctly grasped. Therefore, it is unnecessary to fix the sensor device 1A to the body of the detection target, which widens the degree of freedom of the mountability of the sensor device 1A.

[Configuration of Detector Unit]

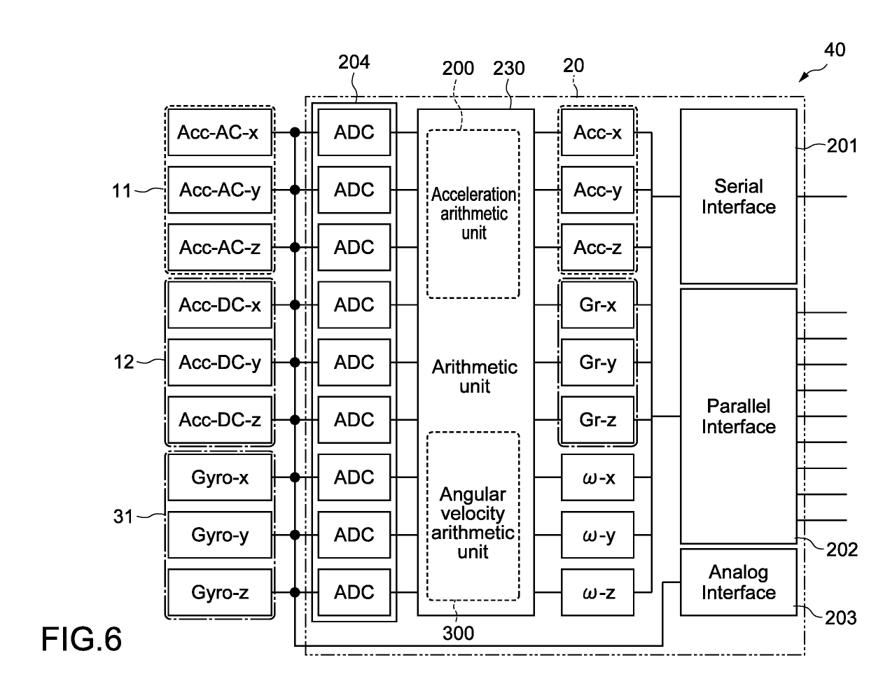

Next, details of the detector unit (inertial sensor) 40 according to this embodiment will be described. FIG. 6 is a block diagram showing a configuration of the detector unit (inertial sensor) 40 according to an embodiment of the present technology.

As shown in FIG. 4, the detector unit (inertial sensor) 40 includes the acceleration sensor element 10, the angular velocity sensor element 30, and the controller 20. Here, the acceleration sensor element 10 and the controller 20 will be mainly described.

The acceleration sensor element 10 of this embodiment is configured as an acceleration sensor that detects accelerations in three-axis directions in the local coordinate system (x, y, and z axes).

In particular, the acceleration sensor element 10 of this embodiment is configured to be capable of extracting dynamic acceleration components and static acceleration components from the respective accelerations in the three-axis directions described above.

Here, the dynamic acceleration component typically means an AC component of the acceleration described above and typically corresponds to a motion acceleration (translational acceleration, centrifugal acceleration, tangential acceleration, or the like) of the object described above. Meanwhile, the static acceleration component typically means a DC component of the acceleration described above and typically corresponds to a gravitational acceleration or an acceleration estimated as a gravitational acceleration.

As shown in FIG. 6, the acceleration sensor element 10 includes two types of acceleration detector units (first acceleration detector unit 11 and second acceleration detector unit 12) that each detect information related to the accelerations in the three-axis directions. The angular velocity sensor element 30 includes an angular velocity detector unit 31.

The first acceleration detector unit 11 is a piezoelectric acceleration sensor and outputs each of a signal (Acc-AC-x) including information associated with an acceleration parallel to the x-axis direction, a signal (Acc-AC-y) including information associated with an acceleration parallel to the y-axis direction, and a signal (Acc-AC-z) including information associated with an acceleration parallel to the z-axis direction. Those signals (first detection signals) each have an alternating-current waveform corresponding to the acceleration of each axis.

Meanwhile, the second acceleration detector unit 12 is a non-piezoelectric acceleration sensor and outputs each of a signal (Acc-DC-x) including information associated with an acceleration parallel to the x-axis direction, a signal (Acc-DC-y) including information associated with an acceleration parallel to the y-axis direction, and a signal (Acc-DC-z) including information associated with an acceleration parallel to the z-axis direction. Those signals (second detection signals) each have an output waveform in which an alternating-current component corresponding to the acceleration of each axis is superimposed on a direct-current component.

The controller 20 includes an acceleration arithmetic unit 200 and an angular velocity arithmetic unit 300. The acceleration arithmetic unit 200 extracts dynamic acceleration components and static acceleration components from the respective accelerations in the three-axis directions described above on the basis of the output of the first acceleration detector unit 11 (first detection signals) and the output of the second acceleration detector unit 12 (second detection signals). The angular velocity arithmetic unit 300 calculates the angular velocity signals about the three axes (ω-x, ω-y, ω-z) (third detection signals) on the basis of the angular velocity detection signals about the three axes (Gyro-x, Gyro-y, Gyro-z), respectively.

It should be noted that the controller 20 may be achieved by hardware elements such as a CPU (Central Processing Unit), a RAM (Random Access Memory), and a ROM (Read Only Memory) used in a computer and necessary software. Instead of or in addition to the CPU, a PLD (Programmable Logic Device) such as a FPGA (Field Programmable Gate Array), a DSP (Digital Signal Processor), or the like may be used.

(Acceleration Sensor Element)

Subsequently, details of the acceleration sensor element 10 constituting the detector unit (inertial sensor) 40 will be described.

FIGS. 7 to 9 are a perspective view of the front surface side, a perspective view of the back surface side, and a plan view of the front surface side schematically showing the configuration of the acceleration sensor element 10, respectively.

The acceleration sensor element 10 includes an element main body 110, the first acceleration detector unit 11 (first detection elements 11x1, 11x2, 11y1, 11y2) and the second acceleration detector unit 12 (second detection elements 12x1, 12x2, 12y1, 12y2).

The element main body 110 includes a main surface portion 111 parallel to the xy plane and a support portion 114 on the opposite side. The element main body 110 is typically constituted of an SOI (Silicon On Insulator) substrate and has a laminated structure including an active layer (silicon substrate), which forms the main surface portion 111, and a frame-shaped support layer (silicon substrate), which forms the support portion 114. The main surface portion 111 and the support portion 114 have thicknesses different from each other, and the support portion 114 is formed to be thicker than the main surface portion 111.

The element main body 110 includes a movable plate 120 (movable portion) capable of moving by reception of an acceleration. The movable plate 120 is provided at the center portion of the main surface portion 111 and is formed by processing the active layer forming the main surface portion 111 into a predetermined shape. More specifically, the movable plate 120 including a plurality of (four in this example) blade portions 121 to 124 each having the shape symmetric with respect to the center portion of the main surface portion 111 is constituted by a plurality of groove portions 112 formed in the main surface portion 111. The circumferential portion of the main surface portion 111 constitutes a base portion 115 that faces the support portion 114 in the z-axis direction.

As shown in FIG. 8, the support portion 114 is formed into a frame including a rectangular recess portion 113 in which the back surface of the movable plate 120 is opened. The support portion 114 is constituted as a joint surface to be joined to a support substrate (not shown in the figure). The support substrate may be constituted of a circuit board that electrically connects the sensor element 10 and the controller 20 or may be constituted of a relay board or package board that is electrically connected to the circuit board. Alternatively, the support portion 114 may include a plurality of external connection terminals electrically connected to the circuit board, the relay board, or the like.

The blade portions 121 to 124 of the movable plate 120 are each constituted of a piece of board having a predetermined shape (substantially hexagonal shape in this example) and are disposed at intervals of 90° about the center axis parallel to the Z axis. The thickness of each of the blade portions 121 to 124 corresponds to the thickness of the above-mentioned active layer constituting the main surface portion 111. The blade portions 121 to 124 are mutually integrally connected at the center portion 120C of the movable plate 120 and are integrated and supported so as to be relatively movable to the base portion 115.

As shown in FIG. 8, the movable plate 120 further includes a weight portion 125. The weight portion 125 is integrally provided to the back surface of the center portion of the movable plate 120 and the back surfaces of the respective blade portions 121 to 124. The size, the thickness, and the like of the weight portion 125 are not particularly limited and are set to have an appropriate size with which desired vibration properties of the movable plate 120 are acquired. The weight portion 125 is formed by, for example, processing the supporting layer forming the support portion 114 into a predetermined shape.

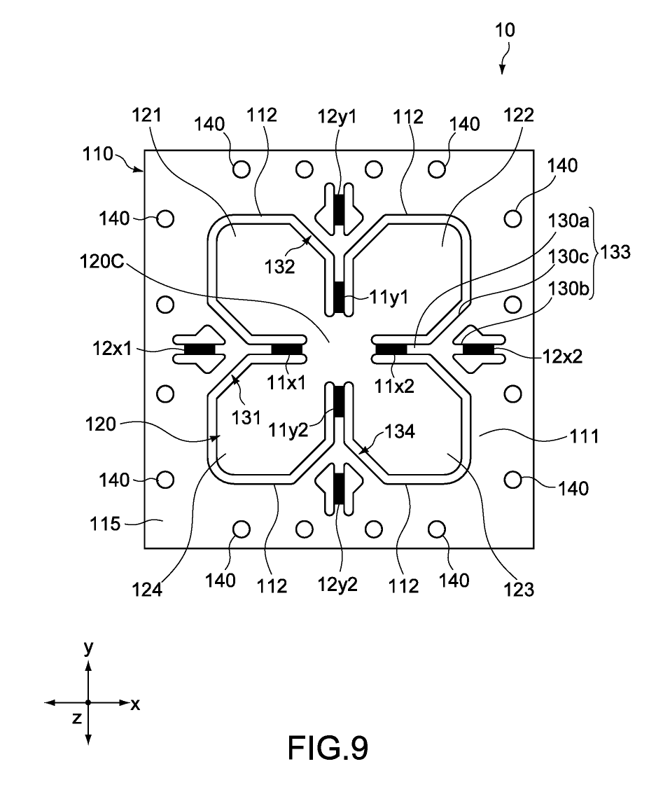

As shown in FIGS. 7 and 9, the movable plate 120 is connected to the base portion 115 via a plurality of (four in this example) bridge portions 131 to 134. The plurality of bridge portions 131 to 134 are each provided between the blade portions 121 to 124 and are formed by processing the active layer forming the main surface portion 111 into a predetermined shape. The bridge portion 131 and the bridge portion 133 are disposed to face each other in the x-axis direction, and the bridge portion 132 and the bridge portion 134 are disposed to face each other in the y-axis direction.

The bridge portions 131 to 134 constitute a part of the movable portion relatively movable to the base portion 115 and elastically support the center portion 120C of the movable plate 120. The bridge portions 131 to 134 each have an identical configuration and each includes, as shown in FIG. 9, a first beam portion 130a, a second beam portion 130b, and a third beam portion 130c.

The first beam portion 130a linearly extends from the circumferential portion of the center portion 120C of the movable plate 120 to each of the x-axis direction and the y-axis direction and is disposed between corresponding two of the blade portions 121 to 124 adjacent to each other. The second beam portion 130b linearly extends in each of the x-axis direction and the y-axis direction and couples the first beam portion 130a and the base portion 115 to each other.

The third beam portion 130c linearly extends in each of directions respectively intersecting with the x-axis direction and the y-axis direction and couples the intermediate portion between the first beam portion 130a and the second beam portion 130b and the base portion 115 to each other. Each of the bridge portions 131 to 134 includes two third beam portions 130c and is configured such that the two third beam portions 130c sandwich the single second beam portion 130b therebetween in the xy plane.

The rigidity of the bridging portions 131 to 134 is set to have an appropriate value at which the movable plate 120 that is moving can be stably supported. In particular, the bridging portions 131 to 134 are set to have appropriate rigidity at which the bridging portions 131 to 134 can be deformed by the self-weight of the movable plate 120. The magnitude of the deformation is not particularly limited as long as it can be detected by the second acceleration detector unit 12 to be described later.

As described above, the movable plate 120 is supported to the base portion 115 of the element main body 110 via the four bridge portions 131 to 134 and is configured to be capable of moving (movable) relative to the base portion 115 by an inertial force corresponding to the acceleration with the bridge portions 131 to 134 being set as a fulcrum.

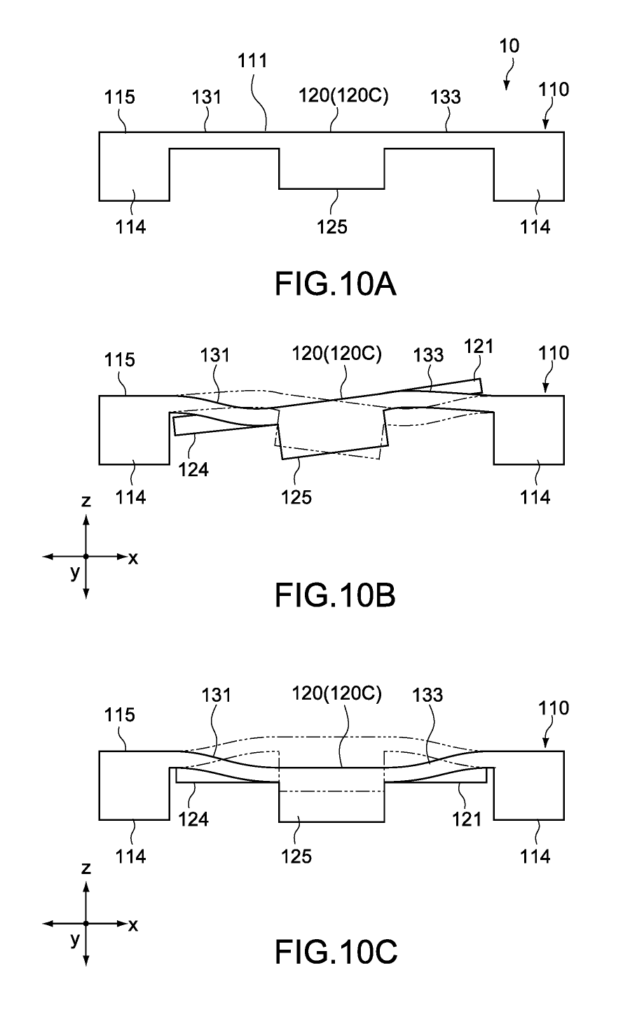

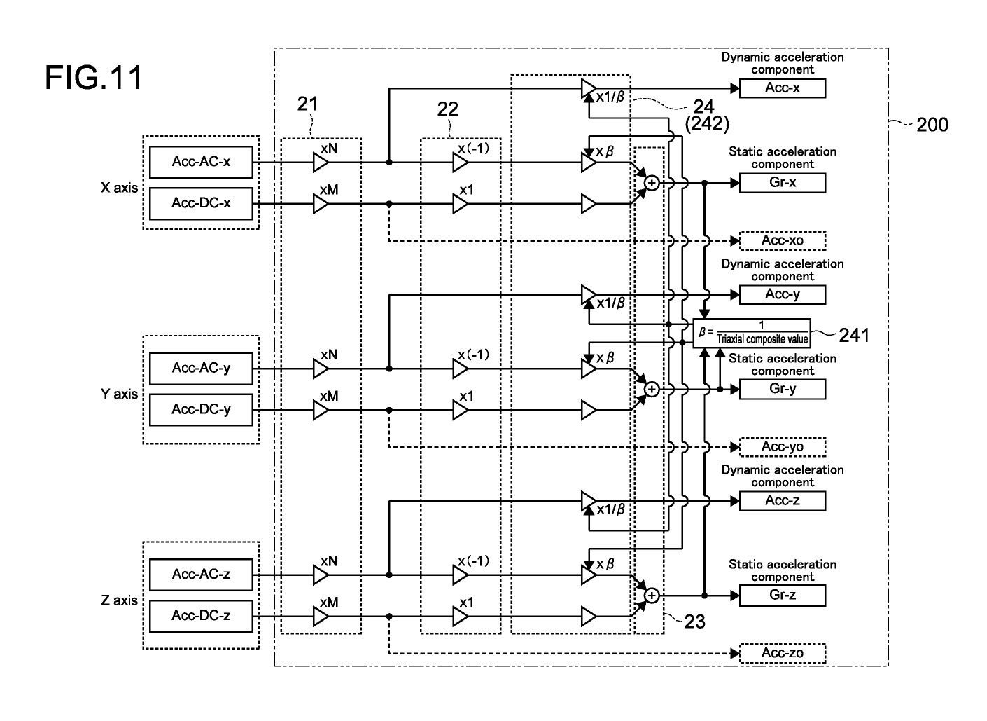

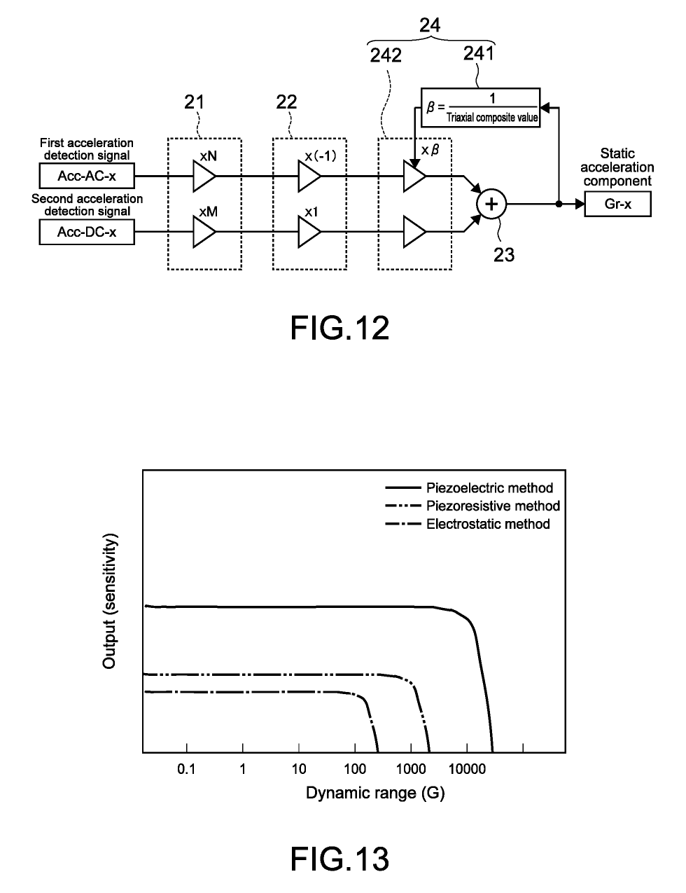

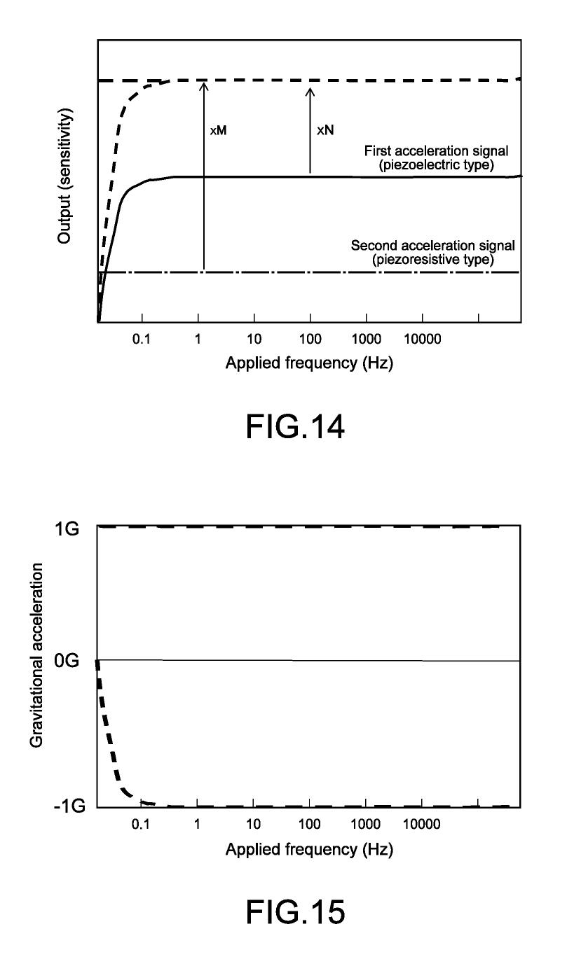

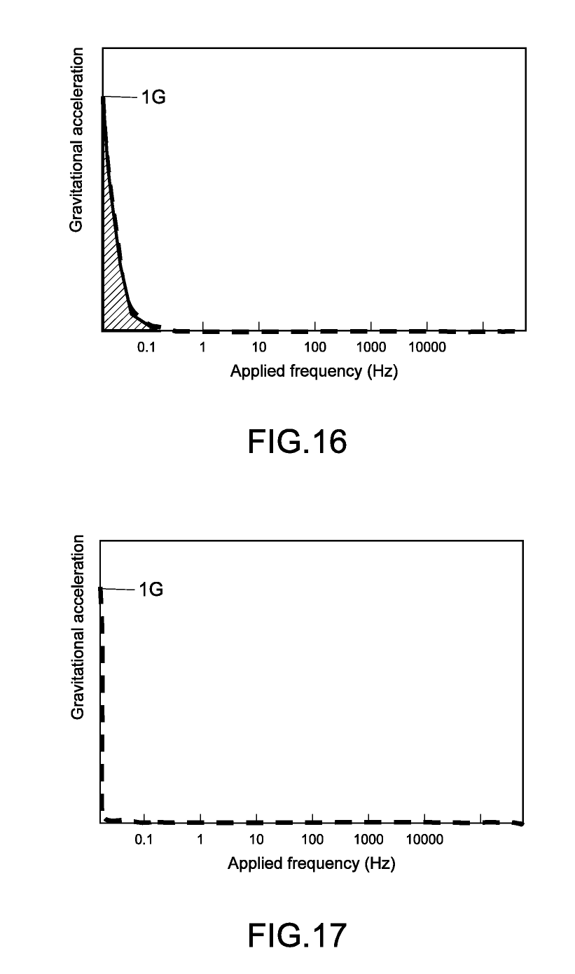

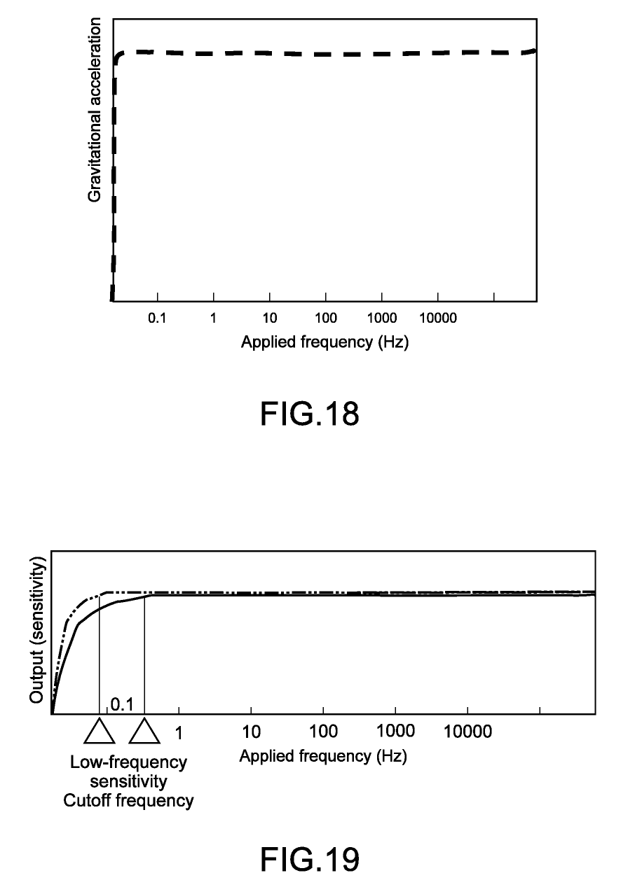

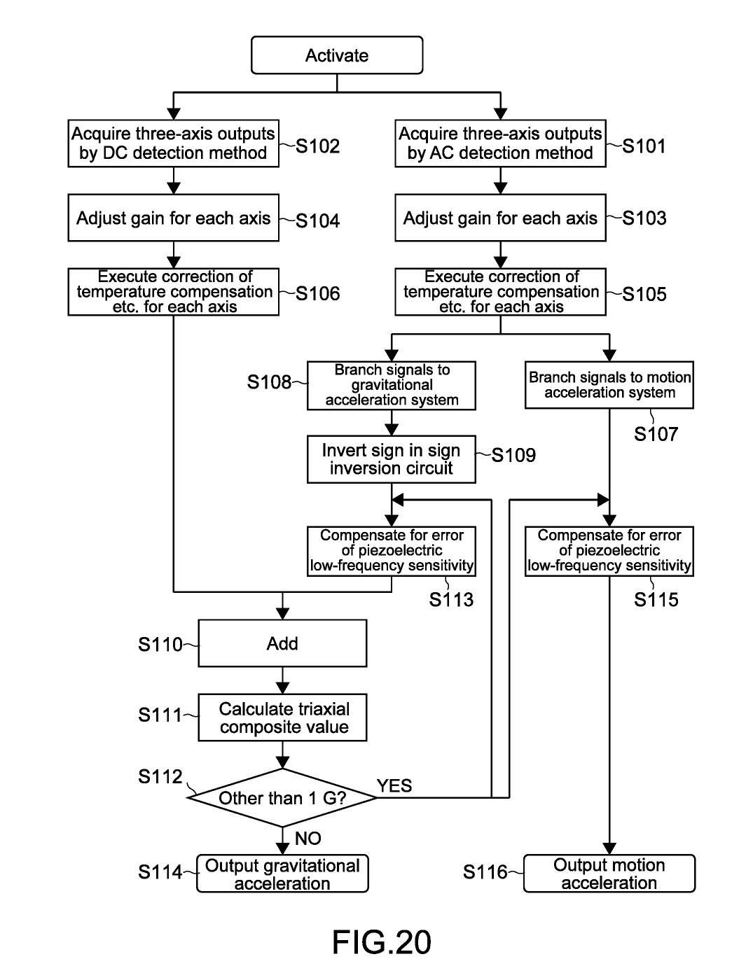

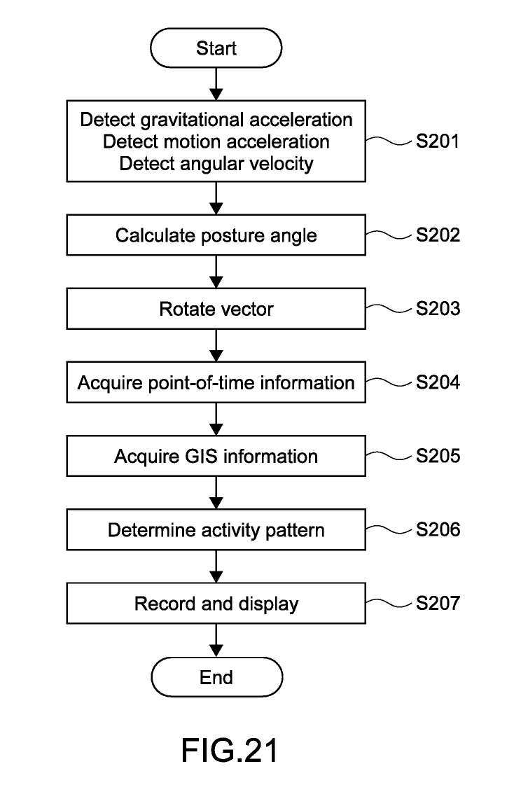

FIGS. 10A to 100 are schematic sectional side views for describing a state of a motion of the movable plate 120, in which A shows a state where accelerations are not applied, B shows a state where the acceleration along the x-axis direction occurs, and C shows a state where the acceleration along the z-axis direction occurs. It should be noted that the solid line in FIG. 10B shows a state where the acceleration occurs in the left direction on the plane of the figure, and the solid line in FIG. 100 shows a state where the acceleration occurs in the upper direction on the plane of the figure.

When accelerations do not occur, as shown in FIGS. 7 and 10A, the movable plate 120 is maintained in a state parallel to the surface of the base portion 115. In this state, for example, when the acceleration along the x-axis direction occurs, as shown in FIG. 10B, the movable plate 120 tilts in the counterclockwise direction about the bridge portions 132 and 134 extending in the y-axis direction. With this configuration, the bridge portions 131 and 133 facing each other in the x-axis direction each receive bending stress in the directions opposite to each other along the z-axis direction.

Similarly, when the acceleration along the y-axis direction occurs, though not shown in the figure, the movable plate 120 tilts in the counterclockwise direction (or clockwise direction) about the bridge portions 131 and 133 extending in the x-axis direction. The bridge portions 132 and 134 facing each other in the y-axis direction each receive bending stress in the directions opposite to each other along the z-axis direction.

Meanwhile, when the acceleration along the z-axis direction occurs, as shown in FIG. 100, the movable plate 120 rises and falls with respect to the base portion 115, and the bridge portions 131 to 134 each receive bending stress in an identical direction along the z-axis direction.

The first acceleration detector unit 11 and the second acceleration detector unit 12 are provided to each of the bridge portions 131 to 134. The detector unit (inertial sensor) 40 detects the deformation resulting from the bending stress of the bridge portions 131 to 134 by the acceleration detector units 11 and 12, and thus measures the direction and magnitude of the acceleration that acts on the sensor element 10.

Hereinafter, details of the acceleration detector units 11 and 12 will be described.

As shown in FIG. 9, the first acceleration detector unit 11 includes a plurality of (four in this example) first detection elements 11x1, 11x2, 11y1, and 11y2.

The detection elements 11x1 and 11x2 are provided on the axial centers of the respective surfaces of the two bridge portions 131 and 133 facing each other in the x-axis direction. One detection element 11x1 is disposed in the first beam portion 130a of the bridge portion 131, and the other detection element 11x2 is disposed in the first beam portion 130a of the bridge portion 133. In contrast to this, the detection elements 11y1 and 11y2 are provided on the axial centers of the respective surfaces of the two bridge portions 132 and 134 facing each other in the y-axis direction. One detection element 11y1 is disposed in the first beam portion 130a of the bridge portion 132, and the other detection element 11y2 is disposed in the first beam portion 130a of the bridge portion 134.

The first detection elements 11x1 to 11y2 each have an identical configuration and, in this embodiment, are each constituted of a rectangular piezoelectric detection element having a long side in the axial direction of the first beam portion 130a. The first detection elements 11x1 to 11y2 are each constituted of a laminate including a lower electrode layer, a piezoelectric film, and an upper electrode layer.

The piezoelectric film is typically made of piezoelectric zirconate titanate (PZT), but the present technology is not limited thereto as a matter of course. The piezoelectric film causes a potential difference, which corresponds to the amount of bending deformation (stress) of the first beam portion 130a in the z-axis direction, between the upper electrode layer and the lower electrode layer (piezoelectric effect). The upper electrode layer is electrically connected to each of the relay terminals 140 provided to the surface of the base portion 115 via a wiring layer (not shown in the figure) formed on the bridge portions 131 to 134. The relay terminal 140 may be configured as an external connection terminal electrically connected to the support substrate described above. For example, a bonding wire, one terminal of which is connected to the support substrate described above, is connected to the relay terminal 140 at the other terminal thereof. The lower electrode layer is typically connected to a reference potential such as a ground potential.

Since the first acceleration detector unit 11 configured as described above performs output only when the stress changes because of the characteristics of the piezoelectric film, and does not perform output in a state where a stress value is not changed even if the stress is applied, the first acceleration detector unit 11 mainly detects the magnitude of the motion acceleration that acts on the movable plate 120. Therefore, the output of the first acceleration detector unit 11 (first detection signal) mainly includes an output signal having an alternating-current waveform that is a dynamic component (AC component) corresponding to the motion acceleration.

Meanwhile, as shown in FIG. 9, the second acceleration detector unit 12 includes a plurality of (four in this example) second detection elements 12x1, 12x2, 12y1, and 12y2.

The detection elements 12x1 and 12x2 are provided on the axial centers of the respective surfaces of the two bridge portions 131 and 133 facing each other in the x-axis direction. One detection element 12x1 is disposed in the second beam portion 130b of the bridge portion 131, and the other detection element 12x2 is disposed in the second beam portion 130b of the bridge portion 133. In contrast to this, the detection elements 12y1 and 12y2 are provided on the axial centers of the respective surfaces of the two bridge portions 132 and 134 facing each other in the y-axis direction. One detection element 12y1 is disposed in the second beam portion 130b of the bridge portion 132, and the other detection element 12y2 is disposed in the second beam portion 130b of the bridge portion 134.

The second detection elements 12x1 to 12y2 each have an identical configuration and, in this embodiment, are each constituted of a piezoresistive detection element having a long side in the axial direction of the second beam portion 130b. The second detection elements 12x1 to 12y2 each include a resistive layer and a pair of terminal portions connected to both ends of the resistive layer in the axial direction.

The resistive layer is a conductor layer that is formed by, for example, doping an impurity element in the surface (silicon layer) of the second beam portion 130b, and causes a resistance change, which corresponds to the amount of bending deformation (stress) of the second beam portion 130b in the z-axis direction, between the pair of terminal portions (piezoresistive effect). The pair of terminal portions is electrically connected to each of the relay terminals 140 provided to the surface of the base portion 115 via a wiring layer (not shown in the figure) formed on the bridge portions 131 to 134.

Since the second acceleration detector unit 12 configured as described above has a resistance value determined by an absolute stress value because of the piezoresistive characteristics, the second acceleration detector unit 12 detects not only the motion acceleration that acts on the movable plate 120 but also the gravitational acceleration that acts on the movable plate 120. Therefore, the output of the second acceleration detector unit 11 (second detection signal) has an output waveform in which a dynamic component (AC component) corresponding to the motion acceleration is superimposed on a gravitational acceleration or a static component (DC component) corresponding to the gravitational acceleration.

It should be noted that the second detection elements 12x1 to 12y2 are not limited to the example in which the second detection elements 12x1 to 12y2 are each constituted of the piezoresistive detection element, and may be each constituted of other non-piezoelectric detection element capable of detecting the acceleration of the DC component, for example, like an electrostatic type. In a case of the electrostatic type, a movable electrode portion and a fixed electrode portion constituting an electrode pair are disposed to face each other in the axial direction of the second beam portion 130b and are configured such that a facing distance between the electrode portions changes depending on the amount of bending deformation of the second beam portion 130b.

The first acceleration detector unit 11 outputs each of the acceleration detection signals in the respective x-axis direction, y-axis direction, and z-axis direction (Acc-AC-x, Acc-AC-y, Acc-AC-z) to the controller 20 on the basis of the outputs of the first detection elements 11x1 to 11y2 (see FIG. 5).

The acceleration detection signal in the x-axis direction (Acc-AC-x) corresponds to a difference signal (ax1-ax2) between the output of the detection element 11x1 (ax1) and the output of the detection element 11x2 (ax2). The acceleration detection signal in the y-axis direction (Acc-AC-y) corresponds to a difference signal (ay1-ay2) between the output of the detection element 11y1 (ay1) and the output of the detection element 11y2 (ay2). Additionally, the acceleration detection signal in the z-axis direction (Acc-AC-z) corresponds to the sum of the outputs of the detection elements 11x1 to 11y2 (ax1+ax2+ay1+ay2).

Similarly, the second acceleration detector unit 12 outputs each of the acceleration detection signals in the respective x-axis direction, y-axis direction, and z-axis direction (Acc-DC-x, Acc-DC-y, Acc-DC-z) to the controller 20 on the basis of the outputs of the second detection elements 12x1 to 12y2 (see FIG. 5).

The acceleration detection signal in the x-axis direction (Acc-DC-x) corresponds to a difference signal (bx1-bx2) between the output of the detection element 12x1 (bx1) and the output of the detection element 12x2 (bx2). The acceleration detection signal in the y-axis direction (Acc-DC-y) corresponds to a difference signal (by1-by2) between the output of the detection element 12y1 (by1) and the output of the detection element 12y2 (by2). Additionally, the acceleration detection signal in the z-axis direction (Acc-DC-z) corresponds to the sum of the outputs of the detection elements 12x1 to 12y2 (bx1+bx2+by1+by2).

The arithmetic processing of the acceleration detection signals in the respective axial directions described above may be executed at a previous stage of the controller unit 50 or may be executed in the controller unit 50.

(Controller)

Subsequently, the controller (signal processing circuit) 20 will be described.

The controller 20 is electrically connected to the acceleration sensor element 10. The controller 20 may be mounted inside a device together with the acceleration sensor element 10 or may be mounted in an external device different from the above-mentioned device. In the former case, for example, the controller 20 may be mounted on a circuit board on which the acceleration sensor element 10 is to be mounted or may be mounted on a substrate different from the above-mentioned circuit board via a wiring cable or the like. In the latter case, for example, the controller 20 is configured to be communicable with the acceleration sensor element 10 wirelessly or wiredly.

The controller 20 includes the acceleration arithmetic unit 200, the angular velocity arithmetic unit 300, a serial interface 201, a parallel interface 202, and an analog interface 203. The controller 20 is electrically connected to controller units of various devices that receive the output of the detector unit (inertial sensor) 40.

The acceleration arithmetic unit 200 extracts each of dynamic acceleration components (Acc-x, Acc-y, Acc-z) and static acceleration components (Gr-x, Gr-y, Gr-z) on the basis of the acceleration detection signals in the respective axial directions, which are output from the first acceleration detector unit 11 and the second acceleration detector unit 12.

It should be noted that the acceleration arithmetic unit 200 is achieved by loading a program, which is recorded in a ROM as an example of a non-transitory computer readable recording medium, to a RAM or the like and executing the program by the CPU.

The angular velocity arithmetic unit 300 calculates angular velocity signals about the three axes (ω-x, ω-y, ω-z) on the basis of the angular velocity detection signals about the three axes (Gyro-x, Gyro-y, Gyro-z), respectively, and outputs those signals to the outside via the serial interface 201, the parallel interface 202, or the analog interface 203. The angular velocity arithmetic unit 300 may be constituted separately from the acceleration arithmetic unit 200 or may be constituted of the arithmetic unit 230 in common with the acceleration arithmetic unit 200.

The serial interface 201 is configured to be capable of sequentially outputting the dynamic and static acceleration components in the respective axes, which are generated in the acceleration arithmetic unit 200, and the angular velocity signals in the respective axes, which are generated in the angular velocity arithmetic unit 300, to the controller units described above. The parallel interface 202 is configured to be capable of outputting the dynamic and static acceleration components in the respective axes, which are generated in the acceleration arithmetic unit 200, to the controller units described above in parallel. The controller 20 may include at least one of the serial interface 201 or the parallel interface 202 or may selectively switch the interface depending on commands from the controller units described above. The analog interface 203 is configured to be capable of outputting the outputs of the first and second acceleration detector units 11 and 12 to the controller units described above without change, but it may be omitted as necessary. It should be noted that FIG. 5 shows converters 204 that analog-digital (AD) convert the acceleration detection signals in the respective axes.

FIG. 11 is a circuit diagram showing a configuration example of the acceleration arithmetic unit 200.

The acceleration arithmetic unit 200 includes a gain adjustment circuit 21, a sign inversion circuit 22, an adder circuit 23, and a correction circuit 24. Those circuits 21 to 24 have a common configuration for each of the x, y, and z axes. The arithmetic processing in common with the respective axes is performed, and the dynamic acceleration components (motion accelerations) and the static acceleration components (gravitational accelerations) in the respective axes are thus extracted.

Hereinafter, representatively, a processing circuit of the acceleration detection signal in the x-axis direction will be described as an example. FIG. 12 shows a processing block that extracts the static acceleration component from the acceleration detection signal in the x-axis direction.

The gain adjustment circuit 21 adjusts gain of each signal such that a first acceleration detection signal (Acc-AC-x) regarding the X-axis direction, which is output from the first acceleration detector unit 11 (11x1, 11x2), and a second acceleration detection signal (Acc-DC-x) regarding x-axis direction, which is output from the second acceleration detector unit 12 (12x1, 12x2), have a level identical to each other. The gain adjustment circuit 21 includes an amplifier that amplifies the output of the first acceleration detector unit 11 (Acc-AC-x) and the output of the second acceleration detector unit 12 (Acc-DC-x).

In general, the output sensitivity and the dynamic range of an acceleration sensor are different depending on a detection method. For example, as shown in FIG. 13, an acceleration sensor in a piezoelectric method has higher output sensitivity and a wider (larger) dynamic range than those of acceleration sensors in a non-piezoelectric method (piezoresistive method, electrostatic method). In this embodiment, the first acceleration detector unit 11 corresponds to an acceleration sensor in a piezoelectric method, and the second acceleration detector unit 12 corresponds to an acceleration sensor in a piezoresistive method.

In this regard, the gain adjustment circuit 21 amplifies the outputs of the acceleration detector units 11 and 12 (first and second acceleration detection signals) by N times and M times, respectively, such that the outputs of those acceleration detector units 11 and 12 have the identical level. The amplification factors N and M are positive numbers and satisfy a relationship where N<M. The values of the amplification factors N and M are not particularly limited and may be set as coefficients that also serve for the temperature compensation of the respective acceleration detector units 11 and 12, depending on an environment of usage (service temperature) of the detector unit (inertial sensor) 40.

FIG. 14 shows an example of the output characteristics of the first acceleration detection signal and the second acceleration detection signal in comparison between the output characteristics before the gain adjustment and the output characteristics after the gain adjustment. In the figure, the horizontal axis represents the frequency of the acceleration that acts on the detector unit (inertial sensor) 40, and the vertical axis represents the output (sensitivity) (the same holds true for FIGS. 15 to 19).

As shown in the figure, in the first acceleration detection signal (Acc-AC-x) in the piezoelectric method, the output sensitivity of the acceleration components in the low-frequency range equal to or smaller than 0.5 Hz is lower than the output sensitivity of the acceleration components in the frequency range higher than the former range, and in particular, the output sensitivity in a static state (motion acceleration is zero) is substantially zero. In contrast to this, the second acceleration detection signal (Acc-DC-x) in the piezoresistive method has constant output sensitivity in the entire frequency range, and thus the acceleration component in the static state (i.e., static acceleration component) can also be detected at constant output sensitivity. Therefore, when the first acceleration detection signal and the second acceleration detection signal are amplified by respective predetermined multiplying factors in the gain adjustment circuit 21 so as to have a level identical to each other, the static acceleration component can be extracted in a difference arithmetic circuit to be described later.

The sign inversion circuit 22 and the adder circuit 23 constitute the difference arithmetic circuit that extracts the static acceleration component (DC component) from the acceleration in each axial direction on the basis of a difference signal between the first acceleration detection signal (Acc-AC-x) and the second acceleration detection signal (Acc-DC-x).

The sign inversion circuit 22 includes an inverting amplifier (amplification factor: −1) that inverts the sign of the first acceleration detection signal (Acc-AC-x) after the gain adjustment. FIG. 15 shows an example of the output characteristics of the first acceleration detection signal (Acc-AC-x) after the sign inversion. Here, a case where the sensor element 10 detects a 1G-acceleration in the x-axis direction is shown as an example.

It should be noted that the second acceleration detection signal (Acc-DC-x) is output to the adder circuit 23 as a subsequent stage, without inverting the sign thereof. The sign inversion circuit 22 may be configured in common with the gain adjustment circuit 21 at the previous stage thereof.

The adder circuit 23 adds the first acceleration detection signal (Acc-AC-x) and the second acceleration detection signal (Acc-DC-x), which are output from the sign inversion circuit 22, and outputs a static acceleration component. FIG. 16 shows an example of the output characteristics of the adder circuit 23. Since the first and second acceleration detection signals are adjusted to have the identical level in the gain adjustment circuit 21, when a difference signal between those signals is obtained, a net static acceleration component (Gr-x) is extracted. The static acceleration component typically corresponds to a gravitational acceleration component or an acceleration component including a gravitational acceleration.

In a case where the static acceleration component output from the adder circuit 23 is only the gravitational acceleration, in theory, the output of a significant acceleration component appears only in the vicinity of 0 Hz as shown in FIG. 17. However, in reality, because of the low detection sensitivity in the vicinity of low frequencies of the piezoelectric-detection-type first acceleration detector unit 11, inevitable superimposition of acceleration components in axial directions (here, y-axis direction and z-axis direction) other than the target axis due to the occurrence of the sensitivity in the other axes, or the like, the dynamic acceleration component in the frequency range hatched in FIG. 16 leaks into the output of the adder circuit 23 as an error component. In this regard, this embodiment includes the correction circuit 24 for cancelling the error on the basis of the output of the adder circuit 23.

The correction circuit 24 includes a triaxial-composite-value arithmetic unit 241 and a low-frequency sensitivity correction unit 242. The correction circuit 24 calculates a correction coefficient β on the basis of the output of the adder circuit 23 (difference signal between first and second acceleration detection signals) and corrects the first acceleration detection signal (Acc-AC-x) by using the correction coefficient β.

The triaxial-composite-value arithmetic unit 241 is provided in common for the processing blocks that extract the static acceleration components in all the x-axis, y-axis, and z-axis directions, and calculates the correction coefficient β by using the total value of the outputs (difference signals between first and second acceleration detection signals) of the adder circuits 23 in the respective axes.

Specifically, the triaxial-composite-value arithmetic unit 241 calculates a composite value (√((Gr-x)²+(Gr-y)²+(Gr-z)²)) of the static acceleration components in the three-axis directions (Gr-x, Gr-y, Gr-z), and while considering a portion exceeding 1 in the composite value as a low-frequency sensitivity error (range hatched in FIG. 15), calculates the correction coefficient β corresponding to the inverse of the composite value described above.

β=1/(√((Gr-x)²+(Gr-y)²+(Gr-z)²))

It should be noted that the values of the static acceleration components in the respective three-axis directions (Gr-x, Gr-y, Gr-z) differ depending on the posture of the acceleration sensor element 10 and further vary from hour to hour according to a change in posture of the acceleration sensor element 10. For example, in a case where the z-axis direction of the acceleration sensor element 10 coincides with the gravity direction (vertical direction), the static acceleration component (Gr-z) in the z-axis direction has the largest value as compared to the static acceleration components (Gr-x, Gr-y) in the x-axis direction and the y-axis direction. In such a manner, the gravity direction of the acceleration sensor element 10 at that point of time can be estimated from the values of the static acceleration components (Gr-x, Gr-y, Gr-z) in the respective three-axis directions.

The low-frequency sensitivity correction unit 242 includes a multiplier that multiplies the first acceleration detection signal (Acc-AC-x) having the inverted sign by the correction coefficient β. With this configuration, the first acceleration detection signal is input to the adder circuit 23 in a state where a low-frequency sensitivity error is reduced, and thus an acceleration signal having the frequency characteristics as shown in FIG. 17 is output from the adder circuit 23. In such a manner, only the static acceleration component corresponding to the gravitational acceleration is output, with the result that the extraction accuracy of the gravitational acceleration component is improved.

In this embodiment, the correction circuit 24 is configured to execute processing of multiplying the first acceleration detection signal by the correction coefficient β when the static acceleration component is calculated, but the present technology is not limited thereto. The correction circuit 24 may be configured to execute processing of multiplying the second acceleration detection signal (Acc-DC-x) by the correction coefficient β or may be configured to switch an acceleration detection signal to be corrected between the first acceleration detection signal and the second acceleration detection signal according to the magnitude of an acceleration change.

In a case where either one of the first acceleration detection signal and the second acceleration detection signal has a predetermined acceleration change or larger, the correction circuit 24 is configured to correct the first acceleration detection signal by using the correction coefficient β. As the acceleration change becomes larger (as a frequency to be applied becomes higher), a proportion at which the error component leaks into the first acceleration detection signal increases, and thus the error component can be effectively reduced. This configuration is particularly effective in a case where the motion acceleration is relatively large, for example, as in a motion analysis application.

Meanwhile, in a case where either one of the first acceleration detection signal and the second acceleration detection signal has a predetermined acceleration change or smaller, the correction circuit 24 is configured to correct the second acceleration detection signal by using the correction coefficient β. As the acceleration change becomes smaller (as a frequency to be applied becomes lower), a proportion at which the error component leaks into the second acceleration detection signal increases, and thus the error component can be effectively reduced. This configuration is particularly effective in a case where the motion acceleration is relatively small, for example, as in a leveling operation of a digital camera.

While the static acceleration components in the respective axial directions are extracted as described above, in order to extract the dynamic acceleration components in the respective axial directions (Acc-x, Acc-y, Acc-z), the first acceleration detection signals (Acc-AC-x, Acc-AC-y, Acc-AC-z), in each of which gain is adjusted in the gain adjustment circuit 21, are referred to as shown in FIG. 11.

Here, the first acceleration detection signal may be used to extract the dynamic acceleration component as it is. However, since there is a case where part of the dynamic acceleration component leaks into the static acceleration component as described above, the dynamic acceleration component is lost and the detection with high accuracy is difficult to perform. In this regard, the first acceleration detection signal is corrected by using the correction coefficient β calculated in the correction circuit 24, so that the detection accuracy of the dynamic acceleration component can be achieved.

More specifically, as shown in FIG. 11, the correction circuit 24 (low-frequency sensitivity correction unit 242) includes a multiplier that multiplies the first acceleration signals (Acc-AC-x, Acc-AC-y, Acc-AC-z) by the inverse (1/β) of the correction coefficient β, which is acquired by the triaxial-composite-value arithmetic unit 241. With this configuration, low-frequency sensitivity components of the first acceleration signals are compensated, and thus the extraction accuracy of the dynamic acceleration components (Acc-x, Acc-y, Acc-z) is improved. FIG. 18 schematically shows the output characteristics of the dynamic acceleration components.

In this embodiment, the correction circuit 24 is configured to execute processing of multiplying the first acceleration detection signal by the inverse (1/β) of the correction coefficient when the dynamic acceleration component is calculated, but the present technology is not limited thereto. The correction circuit 24 may be configured to execute processing of multiplying the second acceleration detection signals (Acc-DC-x, Acc-DC-y, Acc-DC-z) by the inverse (1/β) of the correction coefficient. Alternatively, the correction circuit 24 may be configured to switch an acceleration detection signal to be corrected between the first acceleration detection signal and the second acceleration detection signal according to the magnitude of an acceleration change, as in the case of the above-mentioned calculation technique for the static acceleration components.

The processing of correcting the dynamic acceleration component and the static acceleration component by the low-frequency sensitivity correction unit 242 is typically effective in a case where a composite value calculated in the triaxial-composite-value arithmetic unit 241 is other than 1 G (G: gravitational acceleration). It should be noted that examples of the case where the composite value described above is less than 1 G include a case where the sensor element 10 is in free fall.

It should be noted that the first acceleration detection signal detected by the piezoelectric method has output characteristics like a high-pass filter (HPF), and the output lower than a cutoff frequency thereof remains in the output of the adder circuit 23 as an error component of the low-frequency sensitivity (see FIG. 16). In this embodiment, the error component described above is reduced by an arithmetic technique using the correction circuit 24, but the lower cutoff frequency described above is more desirable in order to enhance the accuracy of cancelling the error component.

In this regard, for example, a piezoelectric body having a relatively large capacitance and internal resistance may be used as the piezoelectric film of each of the detection elements (11x1, 11x2, 11y1, 11y2) constituting the first acceleration detector unit 11. With this configuration, for example, as indicated by a chain line in FIG. 19, the cutoff frequency of the low-frequency sensitivity can be reduced to the vicinity of 0 Hz as much as possible, so that the error component of the low-frequency sensitivity can be made as small as possible.

Next, the method of processing the acceleration signal in the acceleration arithmetic unit 200 configured as described above will be described.

When an acceleration acts on the acceleration sensor element 10, the movable plate 120 moves according to the direction of the acceleration with respect to the base portion 115 in the states shown in FIGS. 10A to 100. The first acceleration detector unit 11 (detection elements 11x1, 11x2, 11y1, 11y2) and the second acceleration detector unit 12 (detection elements 12x1, 12x2, 12y1, 12y2) output detection signals corresponding to the amounts of mechanical deformation of the bridge portions 131 to 134 to the controller 20.

FIG. 20 is a flowchart showing an example of the processing procedure of the acceleration detection signal in the controller 20 (acceleration arithmetic unit 200).

The controller 20 acquires the first acceleration detection signals in the respective axes (Acc-AC-x, Acc-AC-y, Acc-AC-z) from the first acceleration detector unit 11 and receives (acquires) the second acceleration detection signals in the respective axes (Acc-DC-x, Acc-DC-y, Acc-DC-z) from the second acceleration detector unit 12 at predetermined sampling intervals (Steps 101 and 102). Those detection signals may be acquired simultaneously (in parallel) or sequentially (serially).

Sequentially, the controller 20 adjusts gain of each detection signal by the gain adjustment circuit 21 such that the first and second acceleration detection signals have an identical level for each axis (FIG. 14, Steps 103 and 104). Further, as necessary, correction for the purpose of temperature compensation or the like of the first and second acceleration detection signals is performed for each axis (Steps 105 and 106).

Next, the controller 20 branches the first acceleration detection signals in the respective axes (Acc-AC-x, Acc-AC-y, Acc-AC-z) into a dynamic acceleration calculation system (motion acceleration system) and a static acceleration calculation system (gravitational acceleration system) (Steps 107 and 108). The first acceleration detection signal branched to the static acceleration calculation system is input to the adder circuit 23 after the sign thereof is inverted by the sign inversion circuit 22 (FIG. 15, Step 109).

The controller 20 adds the first acceleration detection signals (Acc-AC-x, Acc-AC-y, Acc-AC-z) whose signs are inverted, and the second acceleration detection signals (Acc-DC-x, Acc-DC-y, Acc-DC-z), and calculates static acceleration components (Gr-x, Gr-y, Gr-z) for the respective axes in the adder circuit 23 (FIG. 16, Step 110). Furthermore, the controller 20 calculates a triaxial composite value of those static acceleration components in the triaxial-composite-value arithmetic unit 241 (Step 111) and, in a case where that value is other than 1 G, executes in the low-frequency sensitivity correction unit 242 processing of multiplying the above-mentioned first acceleration detection signals (Acc-AC-x, Acc-AC-y, Acc-AC-z) whose signs are inverted, by the correction coefficient β that is the inverse of the composite value described above (Steps 112 and 113). When the composite value described above is 1 G, the controller 20 outputs the calculated gravitational acceleration components (static acceleration components) to the outside (Step 114). It should be noted that the present technology is not limited to the above, and each time the composite value described above is calculated, the calculated gravitational acceleration components (static acceleration components) may be output to the outside.

Meanwhile, when the composite value described above is other than 1 G, the controller 20 executes the processing of multiplying the first acceleration detection signals (Acc-AC-x, Acc-AC-y, Acc-AC-z), which are branched to the motion acceleration system, by the inverse (1/β) of the calculated correction coefficient β (Steps 112 and 115). When the composite value described above is 1 G, the controller 20 outputs the calculated motion acceleration components (dynamic acceleration components) to the outside (Step 116). It should be noted that the present technology is not limited to the above, and each time the composite value described above is calculated, the calculated motion acceleration components (dynamic acceleration components) may be output to the outside.

As described above, the detector unit (inertial sensor) 40 in this embodiment is configured to use the difference in detection methods for the first and second acceleration detector units 11 and 12 to extract the dynamic acceleration components and the static acceleration components from those outputs. With this configuration, the motion acceleration that acts on the user U as a detection target can be accurately measured.

Further, according to this embodiment, since the gravitational acceleration components can be accurately extracted from the output of the detector unit (inertial sensor) 40, the posture of the detection target with respect to the gravity direction can be highly accurately detected. With this configuration, for example, the horizontal posture of a detection target such as a flight vehicle can be stably maintained.

Furthermore, according to this embodiment, since a piezoelectric acceleration sensor is employed as the first acceleration detector unit 11, and a non-piezoelectric (piezoresistive or electrostatic) acceleration sensor is employed as the second acceleration detector unit 12, an inertial sensor having a wide dynamic range and high sensitivity in a low-frequency range can be obtained.

[Operation of Activity Pattern Recognition System]

Sequentially, a typical operation of the activity pattern recognition system 1 configured as described above will be described with reference to FIGS. 20 and 21. FIG. 21 is a flowchart for describing an operation example of the activity pattern recognition system 1.

When the system is activated by power-on or the like, the sensor device 1A detects, by the detector unit (inertial sensor) 40, the gravitational acceleration components (static acceleration components), the motion acceleration components (dynamic acceleration components), and angular velocity components (ωₓ, ω_{y}, ω_{z}) in the local coordinate system of the sensor device 1A (Step 201). The detected gravitational acceleration components, motion acceleration components, and angular velocity components are output to the controller unit 50.

In Step 201, the detection of the gravitational acceleration components (static acceleration components) and the motion acceleration components (dynamic acceleration components) is performed by separating the first and second acceleration detection signals detected in the acceleration sensor element 10 into the gravitational acceleration components (static acceleration components) and the motion acceleration components (dynamic acceleration components), and this separation is performed by the processing method described above using FIG. 20. Further, the angular velocity components are detected by the angular velocity sensor element 30. It should be noted that the separation or extraction of those dynamic acceleration components and static acceleration components may be executed inside the controller unit 50.

The angular velocity signals (ω-x, ω-y, ω-z) supplied to the controller unit 50 are input to the posture angle calculation unit 51. The posture angle calculation unit 51 calculates the posture angles (θₓ, θ_{y}, θ_{z}) from the angular velocity signals (ω-x, ω-y, ω-z) (Step 202). The calculated posture angles (θₓ, θ_{y}, θ_{z}) are input to the vector rotation unit 52.

The dynamic acceleration components (Acc-x, Acc-y, Acc-z) supplied to the controller unit 50 are input to the vector rotation unit 52. The vector rotation unit 52 performs vector rotation and normalization on the input dynamic acceleration components (Acc-x, Acc-y, Acc-z) and rotational angle components (θₓ, θ_{y}, θ_{z}) with the gravity direction being set as a reference, calculates normalized dynamic accelerations, which are motion accelerations (dynamic accelerations) having no influence of the gravity, and normalized posture angles, which are posture angles having no influence of the gravity, and outputs them to the pattern recognition unit 53 (Step 203).

The point-of-time information acquisition unit 54 acquires a point of time, information of a day of week, information of holiday, information of date, and the like, which are detected by the detector unit 40 of the sensor device 1A, and outputs those pieces of information to the pattern recognition unit 53 (Step 204). Furthermore, the GIS information acquisition unit 56 acquires GIS (Geographic Information System) information, extracts a geo category code on the basis of the GIS information, and outputs the geo category code to the pattern recognition unit 53 (Step 205).

An activity pattern is detected by the motion/state recognition unit 531 on the basis of the normalized dynamic accelerations, the normalized posture angles, the information of the point of time, and the like, which are input to the pattern recognition unit 53. This activity pattern is input to the activity pattern determination unit 532. The activity pattern determination unit 532 determines the class of the activity pattern to be classified by using determination processing based on a learning model on the basis of the activity pattern input from the motion/state recognition unit 531, thus determining the class (Step 206). The pattern recognition unit 53 generates, as a control signal, information in which the determined activity class, the geo category code input from the GIS information acquisition unit 56, and the point of time information input from the point-of-time information acquisition unit 54 are associated with one another, and outputs the information to the transmission/reception unit 101.

The terminal device 1B records the control signal input to the terminal device 1B via the transmission/reception unit 404 of the terminal device 1B and further causes the display unit 407 to display the control signal in a predetermined form, for example, in the form of the activity history (Step 207).

As described above, in this embodiment, since the motion direction and the posture angle of the sensor device in a moving state are detected as relative values based on the gravity direction, the motion or the posture of the detection target is highly accurately detected without receiving the influence of the gravity, and the pattern recognition of the motion of the detection target is facilitated. With this configuration, a characteristic motion of the activity of the user can be grasped from the motion of the sensor device 1A.

According to this embodiment, since the detector unit (inertial sensor) 40 that can substantially separate the dynamic acceleration components and the static acceleration components from each other is provided, the dynamic acceleration components can be selectively extracted. Additionally, when the dynamic acceleration components thus extracted and the posture angles are each normalized with the gravity direction being set as a reference, the normalized dynamic accelerations and the normalized posture angles having no influence of the gravity can be obtained.

The motion of the user, in which the motion of the sensor device 1A itself is substantially cancelled, is reflected in the normalized dynamic accelerations and the normalized posture angles. Therefore, in an activity pattern of the detection target that is detected on the basis of the normalized dynamic accelerations and normalized posture angles as described above, the swing of the sensor device 1A itself is substantially cancelled. Therefore, accurate pattern recognition can be performed. In such a manner, according to this embodiment, only the motion of the detection target can be substantially correctly grasped irrespective of the state in which the sensor device 1A is fixed or not fixed to the detection target.

Hereinabove, the embodiment of the present technology has been described, but the present technology is not limited to the embodiment described above and can be variously modified as a matter of course.

For example, in the embodiment described above, the form in which the sensor device 1A (pendant 3) is hung on the neck of the user has been described as an example, but the present technology is not limited thereto. The sensor device 1A may be hung from the waist with a strap, mounted to the clothing of the user with a clip or the like, or put in a breast pocket. In such cases as well, activity recognition of the user can be determined with high accuracy. Further, even in a case where the sensor device 1A is embedded in clothing or mounted to a hair band or the tip of hair, the action and effect similar to those described above can be obtained.

Alternatively, the sensor device 1A may be put into a bag of the user. Even in a case where the bag is put into a bicycle basket or the like, the sensor device 1A can recognize that the user is riding a bicycle according the tilt of the bicycle.

Further, the sensor device 1A may be mounted to a logistics cargo. In this case, a posture of the sensor device 1A, a force (acceleration) applied to the sensor device 1A, or the like during the transit can be traced.

Further, in the above embodiment, the acceleration sensor element 10 shown in FIGS. 7 to 7 is used as a sensor element, but the configuration is not particularly limited as long as the sensor element can detect the accelerations in the three-axis directions. Similarly, the calculation method of extracting the dynamic acceleration components and the static acceleration components from the accelerations that act on the sensor elements is not also limited to the example described above, and an appropriate calculation technique can be employed.

It should be noted that the present technology can also have the following configurations.

(1) An information processing apparatus, including
a controller unit that
   calculates, on a basis of a dynamic acceleration component and a static acceleration component of a detection target moving within a space that are extracted from an acceleration in each direction of three axes of the detection target, a temporal change of the dynamic acceleration component with respect to the static acceleration component, and
   determines a motion of the detection target on a basis of the temporal change of the dynamic acceleration component.
      (2) The information processing apparatus according to (1), in which
the controller unit includes
   an arithmetic unit that calculates a normalized dynamic acceleration obtained by normalizing the dynamic acceleration component in a gravity direction, and
   a pattern recognition unit that determines the motion of the detection target on a basis of the normalized dynamic acceleration.
      (3) The information processing apparatus according to (2), in which
the arithmetic unit further calculates a posture angle of the detection target on a basis of information related to an angular velocity about each of the three axes, and
the pattern recognition unit determines the motion of the detection target on a basis of the normalized dynamic acceleration and the posture angle.
   (4) The information processing apparatus according to (2) or (3), in which
the pattern recognition unit determines an activity class of the detection target on a basis of the motion of the detection target.
   (5) The information processing apparatus according to any one of (1) to (4), further including
a detector unit that is attached to the detection target and detects the acceleration.
   (6) The information processing apparatus according to (5), in which
the detector unit includes an acceleration arithmetic unit that extracts the dynamic acceleration component and the static acceleration component for each direction of the three axes on a basis of a first detection signal having an alternating-current waveform corresponding to the acceleration and a second detection signal having an output waveform in which an alternating-current component corresponding to the acceleration is superimposed on a direct-current component.
   (7) The information processing apparatus according to (6), in which
the acceleration arithmetic unit includes an arithmetic circuit that extracts the static acceleration component from the acceleration on a basis of a difference signal between the first detection signal and the second detection signal.
   (8) The information processing apparatus according to (7), in which
the acceleration arithmetic unit further includes a gain adjustment circuit that adjusts gain of each signal such that the first detection signal and the second detection signal have an identical level.
   (9) The information processing apparatus according to (7) or (8), in which
the acceleration arithmetic unit further includes a correction circuit that calculates a correction coefficient on a basis of the difference signal and corrects one of the first detection signal and the second detection signal by using the correction coefficient.
   (10) The information processing apparatus according to any one of (5) to (9), in which
the detector unit is portable without being fixed to the detection target.
   (11) The information processing apparatus according to any one of (5) to (10), in which
the detector unit includes a sensor element including
   an element main body that includes a movable portion movable by reception of an acceleration,
   a piezoelectric first acceleration detector unit that outputs a first detection signal including information related to the acceleration in each direction of the three axes that acts on the movable portion, and
   a non-piezoelectric second acceleration detector unit that outputs a second detection signal including information related to the acceleration in each direction of the three axes that acts on the movable portion.
      (12) The information processing apparatus according to (11), in which
the second acceleration detector unit includes a piezoresistive acceleration detection element that is provided to the movable portion.
   (13) The information processing apparatus according to (11), in which
the second acceleration detector unit includes an electrostatic acceleration detection element that is provided to the movable portion.

### REFERENCE SIGNS LIST

1 activity pattern recognition system (information processing system)
1A sensor device
1B terminal device
3 pendant
10 acceleration sensor element
11 first acceleration detector unit
12 second acceleration detector unit
40 detector unit (inertial sensor)
50 controller unit
20 controller
110 element main body
120 movable plate (movable portion)
200 acceleration arithmetic unit

1. An information processing apparatus, comprising
a controller unit that
calculates, on a basis of a dynamic acceleration component and a static acceleration component of a detection target moving within a space that are extracted from an acceleration in each direction of three axes of the detection target, a temporal change of the dynamic acceleration component with respect to the static acceleration component, and
determines a motion of the detection target on a basis of the temporal change of the dynamic acceleration component.

2. The information processing apparatus according to claim 1, wherein
the controller unit includes
an arithmetic unit that calculates a normalized dynamic acceleration obtained by normalizing the dynamic acceleration component in a gravity direction, and
a pattern recognition unit that determines the motion of the detection target on a basis of the normalized dynamic acceleration.

3. The information processing apparatus according to claim 2, wherein
the arithmetic unit further calculates a posture angle of the detection target on a basis of information related to an angular velocity about each of the three axes, and
the pattern recognition unit determines the motion of the detection target on a basis of the normalized dynamic acceleration and the posture angle.

4. The information processing apparatus according to claim 2, wherein
the pattern recognition unit determines an activity class of the detection target on a basis of the motion of the detection target.

5. The information processing apparatus according to claim 1, further comprising
a detector unit that is attached to the detection target and detects the acceleration.

6. The information processing apparatus according to claim 5, wherein
the detector unit includes an acceleration arithmetic unit that extracts the dynamic acceleration component and the static acceleration component for each direction of the three axes on a basis of a first detection signal having an alternating-current waveform corresponding to the acceleration and a second detection signal having an output waveform in which an alternating-current component corresponding to the acceleration is superimposed on a direct-current component.

7. The information processing apparatus according to claim 6, wherein
the acceleration arithmetic unit includes an arithmetic circuit that extracts the static acceleration component from the acceleration on a basis of a difference signal between the first detection signal and the second detection signal.

8. The information processing apparatus according to claim 7, wherein
the acceleration arithmetic unit further includes a gain adjustment circuit that adjusts gain of each signal such that the first detection signal and the second detection signal have an identical level.

9. The information processing apparatus according to claim 7, wherein
the acceleration arithmetic unit further includes a correction circuit that calculates a correction coefficient on a basis of the difference signal and corrects one of the first detection signal and the second detection signal by using the correction coefficient.

10. The information processing apparatus according to claim 5, wherein
the detector unit is configured to be portable without being fixed to the detection target.

11. The information processing apparatus according to claim 5, wherein
the detector unit includes a sensor element including
an element main body that includes a movable portion movable by reception of an acceleration,
a piezoelectric first acceleration detector unit that outputs a first detection signal including information related to the acceleration in each direction of the three axes that acts on the movable portion, and
a non-piezoelectric second acceleration detector unit that outputs a second detection signal including information related to the acceleration in each direction of the three axes that acts on the movable portion.

12. The information processing apparatus according to claim 11, wherein
the second acceleration detector unit includes a piezoresistive acceleration detection element that is provided to the movable portion.

13. The information processing apparatus according to claim 11, wherein
the second acceleration detector unit includes an electrostatic acceleration detection element that is provided to the movable portion.
